SEQUENCE LISTING (23 sequences)

SEQ ID NO: 1 - protein, 1012 aa, Homo sapiens
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFASIISLSLIFYYSKKGRRSYK

SEQ ID NO: 2 - protein, 70 aa, Homo sapiens
EAEEDGDLQCLCVKTTSQVRPRHITSLEVIKAGPHCPTAQLIATLKNGRKICLDLQAPLYKKIIKKLLES

SEQ ID NO: 3 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 4 - protein, 667 aa, Homo sapiens
MAGMKTASGDYIDSSWELRVFVGEEDPEAESVTLRVTGESHIGGVLLKIVEQINRKQDWSDHAIWWEQKRQWLLQTHWTLDKYGILADARLFFGPQHRPVILRLPNRRALRLRASFSQPLFQAVAAICRLLSIRHPEELSLLRAPEKKEKKKKEKEPEEELYDLSKVVLAGGVAPALFRGMPAHFSDSAQTEACYHMLSRPQPPPDPLLLQRLPRPSSLSDKTQLHSRWLDSSRCLMQQGIKAGDALWLRFKYYSFFDLDPKTDPVRLTQLYEQARWDLLLEEIDCTEEEMMVFAALQYHINKLSQSGEVGEPAGTDPGLDDLDVALSNLEVKLEGSAPTDVLDSLTTIPELKDHLRIFRIPRRPRKLTLKGYRQHWVVFKETTLSYYKSQDEAPGDPIQQLNLKGCEVVPDVNVSGQKFCIKLLVPSPEGMSEIYLRCQDEQQYARWMAGCRLASKGRTMADSSYTSEVQAILAFLSLQRTGSGGPGNHPHGPDASAEGLNPYGLVAPRFQRKFKAKQLTPRILEAHQNVAQLSLAEAQLRFIQAWQSLPDFGISYVMVRFKGSRKDEILGIANNRLIRIDLAVGDVVKTWRFSNMRQWNVNWDIRQVAIEFDEHINVAFSCVSASCRIVHEYIGGYIFLSTRERARGEELDEDLFLQLTGGHEAF

SEQ ID NO: 5 - protein, 2541 aa, Homo sapiens
MVALSLKISIGNVVKTMQFEPSTMVYDACRIIRERIPEAPAGPPSDFGLFLSDDDPKKGIWLEAGKALDYYMLRNGDTMEYRKKQRPLKIRMLDGTVKTIMVDDSKTVTDMLMTICARIGITNHDEYSLVRELMEEKKEEITGTLRKDKTLLRDEKKMEKLKQKLHTDDELNWLDHGRTLREQGVEEHETLLLRRKFFYSDQNVDSRDPVQLNLLYVQARDDILNGSHPVSFDKACEFAGFQCQIQFGPHNEQKHKAGFLDLKDFLPKEYVKQKGERKIFQAHKNCGQMSEIEAKVRYVKLARSLKTYGVSFFLVKEKMKGKNKLVPRLLGITKECVMRVDEKTKEVIQEWNLTNIKRWAASPKSFTLDFGDYQDGYYSVQTTEGEQIAQLIAGYIDIILKKKKSKDHFGLEGDEESTMLEDSVSPKKSTVLQQQYNRVGKVEHGSVALPAIMRSGASGPENFQVGSMPPAQQQITSGQMHRGHMPPLTSAQQALTGTINSSMQAVQAAQATLDDFDTLPPLGQDAASKAWRKNKMDESKHEIHSQVDAITAGTASVVNLTAGDPAETDYTAVGCAVTTISSNLTEMSRGVKLLAALLEDEGGSGRPLLQAAKGLAGAVSELLRSAQPASAEPRQNLLQAAGNVGQASGELLQQIGESDTDPHFQDALMQLAKAVASAAAALVLKAKSVAQRTEDSGLQTQVIAAATQCALSTSQLVACTKVVAPTISSPVCQEQLVEAGRLVAKAVEGCVSASQAATEDGQLLRGVGAAATAVTQALNELLQHVKAHATGAGPAGRYDQATDTILTVTENIFSSMGDAGEMVRQARILAQATSDLVNAIKADAEGESDLENSRKLLSAAKILADATAKMVEAAKGAAAHPDSEEQQQRLREAAEGLRMATNAAAQNAIKKKLVQRLEHAAKQAAASATQTIAAAQHAASTPKASAGPQPLLVQSCKAVAEQIPLLVQGVRGSQAQPDSPSAQLALIAASQSFLQPGGKMVAAAKASVPTIQDQASAMQLSQCAKNLGTALAELRTAAQKAQEACGPLEMDSALSVVQNLEKDLQEVKAAARDGKLKPLPGETMEKCTQDLGNSTKAVSSAIAQLLGEVAQGNENYAGIAARDVAGGLRSLAQAARGVAALTSDPAVQAIVLDTASDVLDKASSLIEEAKKAAGHPGDPESQQRLAQVAKAVTQALNRCVSCLPGQRDVDNALRAVGDASKRLLSDSLPPSTGTFQEAQSRLNEAAAGLNQAATELVQASRGTPQDLARASGRFGQDFSTFLEAGVEMAGQAPSQEDRAQVVSNLKGISMSSSKLLLAAKALSTDPAAPNLKSQLAAAARAVTDSINQLITMCTQQAPGQKECDNALRELETVRELLENPVQPINDMSYFGCLDSVMENSKVLGEAMTGISQNAKNGNLPEFGDAISTASKALCGFTEAAAQAAYLVGVSDPNSQAGQQGLVEPTQFARANQAIQMACQSLGEPGCTQAQVLSAATIVAKHTSALCNSCRLASARTTNPTAKRQFVQSAKEVANSTANLVKTIKALDGAFTEENRAQCRAATAPLLEAVDNLSAFASNPEFSSIPAQISPEGRAAMEPIVISAKTMLESAGGLIQTARALAVNPRDPPSWSVLAGHSRTVSDSIKKLITSMRDKAPGQLECETAIAALNSCLRDLDQASLAAVSQQLAPREGISQEALHTQMLTAVQEISHLIEPLANAARAEASQLGHKVSQMAQYFEPLTLAAVGAASKTLSHPQQMALLDQTKTLAESALQLLYTAKEAGGNPKQAAHTQEALEEAVQMMTEAVEDLTTTLNEAASAAGVVGGMVDSITQAINQLDEGPMGEPEGSFVDYQTTMVRTAKAIAVTVQEMVTKSNTSPEELGPLANQLTSDYGRLASEAKPAAVAAENEEIGSHIKHRVQELGHGCAALVTKAGALQCSPSDAYTKKELIECARRVSEKVSHVLAALQAGNRGTQACITAASAVSGIIADLDTTIMFATAGTLNREGTETFADHREGILKTAKVLVEDTKVLVQNAAGSQEKLAQAAQSSVATITRLADVVKLGAASLGAEDPETQVVLINAVKDVAKALGDLISATKAAAGKVGDDPAVWQLKNSAKVMVTNVTSLLKTVKAVEDEATKGTRALEATTEHIRQELAVFCSPEPPAKTSTPEDFIRMTKGITMATAKAVAAGNSCRQEDVIATANLSRRAIADMLRACKEAAYHPEVAPDVRLRALHYGRECANGYLELLDHVLLTLQKPSPELKQQLTGHSKRVAGSVTELIQAAEAMKGTEWVDPEDPTVIAENELLGAAAAIEAAAKKLEQLKPRAKPKEADESLNFEEQILEAAKSIAAATSALVKAASAAQRELVAQGKVGAIPANALDDGQWSQGLISAARMVAAATNNLCEAANAAVQGHASQEKLISSAKQVAASTAQLLVACKVKADQDSEAMKRLQAAGNAVKRASDNLVKAAQKAAAFEEQENETVVVKEKMVGGIAQIIAAQEEMLRKERELEEARKKLAQIRQQQYKFLPSELRDEH

SEQ ID NO: 6 - protein, 381 aa, Homo sapiens
MPFGNTHNKFKLNYKPEEEYPDLSKHNNHMAKVLTLELYKKLRDKETPSGFTVDDVIQTGVDNPGHPFIMTVGCVAGDEESYEVFKELFDPIISDRHGGYKPTDKHKTDLNHENLKGGDDLDPNYVLSSRVRTGRSIKGYTLPPHCSRGERRAVEKLSVEALNSLTGEFKGKYYPLKSMTEKEQQQLIDDHFLFDKPVSPLLLASGMARDWPDARGIWHNDNKSFLVWVNEEDHLRVISMEKGGNMKEVFRRFCVGLQKIEEIFKKAGHPFMWNQHLGYVLTCPSNLGTGLRGGVHVKLAHLSKHPKFEEILTRLRLQKRGTGGVDTAAVGSVFDVSNADRLGSSEVEQVQLVVDGVKLMVEMEKKLEKGQSIDDMIPAQK

SEQ ID NO: 7 - protein, 335 aa, Homo sapiens
MGKVKVGVNGFGRIGRLVTRAAFNSGKVDIVAINDPFIDLNYMVYMFQYDSTHGKFHGTVKAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASKE

SEQ ID NO: 8 - protein, 462 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 9 - protein, 330 aa, Homo sapiens [sequence omitted]

SEQ ID NO: 10 - protein, 451 aa, Homo sapiens
MRECISIHVGQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKEIIDLVLDRIRKLADQCTGLQGFLVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLIGQIVSSITASLRFDGALNVDLTEFQTNLVPYPRIHFPLATYAPVISAEKAYHEQLSVAEITNACFEPANQMVKCDPRHGKYMACCLLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGVDSVEGEGEEEGEEY

SEQ ID NO: 11 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 12 - protein, 526 aa, Homo sapiens
ADSRDPASDQMQHWKEQRAAQKADVLTTGAGNPVGDKLNVITVGPRGPLLVQDVVFTDEMAHFDRERIPERVVHAKGAGAFGYFEVTHDITKYSKAKVFEHIGKKTPIAVRFSTVAGESGSADTVRDPRGFAVKFYTEDGNWDLVGNNTPIFFIRDPILFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLSQEDPDYGIRDLFNAIATGKYPSWTFYIQVMTFNQAETFPFNPFDLTKVWPHKDYPLIPVGKLVLNRNPVNYFAEVEQIAFDPSNMPPGIEASPDKMLQGRLFAYPDTHRHRLGPNYLHIPVNCPYRARVANYQRDGPMCMQDNQGGAPNYYPNSFGAPEQQPSALEHSIQYSGEVRRFNTANDDNVTQVRAFYVNVLNEEQRKRLCENIAGHLKDAQIFIQKKAVKNFTEVHPDYGSHIQALLDKYNAEKPKNAIHTFVQSGSHLAAREKANL

SEQ ID NO: 13 - protein, 375 aa, Homo sapiens
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 14 - protein, 147 aa, Mus sp.
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 15 - protein, 493 aa, Homo sapiens
MALFGALFLALLAGAHAEFPGCKIRVTSKALELVKQEGLRFLEQELETITIPDLRGKEGHFYYNISEVKVTELQLTSSELDFQPQQELMLQITNASLGLRFRRQLLYWFFYDGGYINASAEGVSIRTGLELSRDPAGRMKVSNVSCQASVSRMHAAFGGTFKKVYDFLSTFITSGMRFLLNQQICPVLYHAGTVLLNSLLDTVPVRSSVDELVGIDYSLMKDPVASTSNLDMDFRGAFFPLTERNWSLPNRAVEPQLQEEERMVYVAFSEFFFDSAMESYFRAGALQLLLVGDKVPHDLDMLLRATYFGSIVLLSPAVIDSPLKLELRVLAPPRCTIKPSGTTISVTASVTIALVPPDQPEVQLSSMTMDARLSAKMALRGKALRTQLDLRRFRIYSNHSALESLALIPLQAPLKTMLQIGVMPMLNERTWRGVQIPLPEGINFVHEVVTNHAGFLTIGADLHFAKGLREVIEKNRPADVRASTAPTPSTAAV

SEQ ID NO: 16 - protein, 1744 aa, Homo sapiens
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAATAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRGSSTWLTAFVLKVLSLAQEQVGGSPEKLQETSNWLLSQQQADGSFQDLSPVIHRSMQGGLVGNDETVALTAFVTIALHHGLAVFQDEGAEPLKQRVEASISKASSFLGEKASAGLLGAHAAAITAYALTLTKAPADLRGVAHNNLMAMAQETGDNLYWGSVTGSQSNAVSPTPAPRNPSDPMPQAPALWIETTAYALLHLLLHEGKAEMADQAAAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 17 - protein, 434 aa, Homo sapiens
MAMQKIFAREILDSRGNPTVEVDLHTAKGRFRAAVPSGASTGIYEALELRDGDKGRYLGKGVLKAVENINNTLGPALLQKKLSVVDQEKVDKFMIELDGTENKSKFGANAILGVSLAVCKAGAAEKGVPLYRHIADLAGNPDLILPVPAFNVINGGSHAGNKLAMQEFMILPVGASSFKEAMRIGAEVYHHLKGVIKAKYGKDATNVGDEGGFAPNILENNEALELLKTAIQAAGYPDKVVIGMDVAASEFYRNGKYDLDFKSPDDPARHITGEKLGELYKSFIKNYPVVSIEDPFDQDDWATWTSFLSGVNIQIVGDDLTVTNPKRIAQAVEKKACNCLLLKVNQIGSVTESIQACKLAQSNGWGVMVSHRSGETEDTFIADLVVGLCTGQIKTGAPCRSERLAKYNQLMRIEEALGDKAIFAGRKFRNPKAK

SEQ ID NO: 18 - protein, 118 aa, Mus sp.
EVQLQQSGPELVKPGASVKISCKASGYTFTDYYMNWVKQSHGKSLEWIGDINPNNGGTSYNQKFKGKATLTVDKSSSATYMELRSLTSEDSAVYYCARGYGYDPFDVWGTGTTVTVSS

SEQ ID NO: 19 - protein, 830 aa, Homo sapiens
MANCQIAILYQRFQRVVFGISQLLCFSALISELTNQKEVAAWTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCKAVQCQHLEAPSEGTMDCVHPLTAFAYGSSCKFECQPGYRVRGLDMLRCIDSGHWSAPLPTCEAISCEPLESPVHGSMDCSPSLRAFQYDTNCSFRCAEGFMLRGADIVRCDNLGQWTAPAPVCQALQCQDLPVPNEARVNCSHPFGAFRYQSVCSFTCNEGLLLVGASVLQCLATGNWNSVPPECQAIPCTPLLSPQNGTMTCVQPLGSSSYKSTCQFICDEGYSLSGPERLDCTRSGRWTDSPPMCEAIKCPELFAPEQGSLDCSDTRGEFNVGSTCHFSCDNGFKLEGPNNVECTTSGRWSATPPTCKGIASLPTPGLQCPALTTPGQGTMYCRHHPGTFGFNTTCYFGCNAGFTLIGDSTLSCRPSGQWTAVTPACRAVKCSELHVNKPIAMNCSNLWGNFSYGSICSFHCLEGQLLNGSAQTACQENGHWSTTVPTCQAGPLTIQEALTYFGGAVASTIGLIMGGTLLALLRKRFRQKDDGKCPLNPHSHLGTYGVFTNAAFDPSP

SEQ ID NO: 20 - protein, 15 aa, Homo sapiens
DTTLSQFTDLNVYLW

SEQ ID NO: 21 - protein, 15 aa, Homo sapiens
SQLTKPISSLTKPYH

SEQ ID NO: 22 - protein, 1039 aa, Homo sapiens
MARALCPLQALWLLEWVLLLLGPCAAPPAWALNLDPVQLTFYAGPNGSQFGFSLDFHKDSHGRVAIVVGAPRTLGPSQEETGGVFLCPWRAEGGQCPSLLFDLRDETRNVGSQTLQTFKARQGLGASVVSWSDVIVACAPWQHWNVLEKTEEAEKTPVGSCFLAQPESGRRAEYSPCRGNTLSRIYVENDFSWDKRYCEAGFSSVVTQAGELVLGAPGGYYFLGLLAQAPVADIFSSYRPGILLWHVSSQSLSFDSSNPEYFDGYWGYSVAVGEFDGDLNTTEYVVGAPTWSWTLGAVEILDSYYQRLHRLRGEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQDSLNPAVKSCVLPQTKTPVSCFNIQMCVGATGHNIPQKLSLNAELQLDRQKPRQGRRVLLLGSQQAGTTLNLDLGGKHSPICHTTMAFLRDEADFRDKLSPIVLSLNVSLPPTEAGMAPAVVLHGDTHVQEQTRIVLDCGEDDVCVPQLQLTASVTGSPLLVGADNVLELQMDAANEGEGAYEAELAVHLPQGAHYMRALSNVEGFERLICNQKKENETRVVLCELGNPMKKNAQIGIAMLVSVGNLEEAGESVSFQLQIRSKNSQNPNSKIVLLDVPVRAEAQVELRGNSFPASLVVAAEEGEREQNSLDSWGPKVEHTYELHNNGPGTVNGLHLSIHLPGQSQPSDLLYILDIQPQGGLQCFPQPPVNPLKVDWGLPIPSPSPIHPAHHKRDRRQIFLPEPEQPSRLQDPVLVSCDSAPCTVVQCDLQEMARGQRAMVTVLAFLWLPSLYQRPLDQFVLQSHAWFNVSSLPYAVPPLSLPRGEAQVWTQLLRALEERAIPIWWVLVGVLGGLLLLTILVLAMWKVGFFKRNRPPLEEDDEEGE

SEQ ID NO: 23 - protein, 16 aa, Artificial Sequence
  Description of Artificial Sequence Synthetic His tag
  SITE (1)..(16): This sequence may encompass 4-16 residues
HHHHHHHHHHHHHHHH

(19) United States
(12) Patent Application Publication
(10) Pub. No.: US 2023/0123357 A1
(43) Pub. Date: Apr. 20, 2023

(54) USE OF DOWNSTREAM FACTORS IN THE KLOTHO PATHWAY TO ASSESS KLOTHO ACTIVITY

(71) Applicant: The Regents of the University of California, Oakland, CA (US)

(72) Inventor: DENA DUBAL, OAKLAND, CA (US)

(73) Assignee: The Regents of the University of California, Oakland, CA (US)

(21) Appl. No.: 17/908,512

(22) PCT Filed: Mar. 3, 2021
(86) PCT No.: PCT/US2021/020706

§ 371 Date: Aug. 31, 2022

Related U.S. Application Data
(60) Provisional application No. 62/984,944, filed on Mar. 4, 2020.

(51) Int. Cl. G01N 33/49; G01N 15/14

(52) U.S. Cl. CPC G01N 33/491; G01N 15/1459; G01N 2015/0084

(57) ABSTRACT

Methods of monitoring Klotho activity are provided.

### CROSS-REFERENCE TO RELATED PATENT APPLICATIONS

The present application is a 371 National Phase Application of International Application No. PCT/US2021/020706 filed Mar. 3, 2021, which claims benefit of priority to U.S. Provisional Patent Application No. 62/984,944, filed Mar. 4, 2020, each of which are incorporated by reference for all purposes.

### SEQUENCE LISTING

The instant application contains a Sequence Listing which has been submitted electronically in ASCII format and is hereby incorporated by reference in its entirety. Said ASCII copy, created on Oct. 3, 2022, is named 081906-1346307-238110US_SL.txt and is 120,630 bytes in size.

### BACKGROUND OF THE INVENTION

The Klotho protein has been described for use in various clinical settings, including for improving cognition (e.g., U.S. Pat. No. 10,300,117) and kidney disease (e.g., Zhou, et al., BMC Nephrol. 2018; 19: 285) among other diseases.

### BRIEF SUMMARY OF THE INVENTION

In some embodiments, methods of assessing activity of a Klotho polypeptide in an animal are provided. In some embodiments, the method comprises administering the Klotho polypeptide to the animal, and then obtaining a sample from the animal and measuring a quantity or activity of any one or more polypeptide of Table 1 or Table 2. In some embodiments, the sample comprises platelets. In some embodiments, the obtaining comprises purifying platelets from blood from the animal. In some embodiments, the method comprises comparing the quantity or activity to a control value (in some embodiments, this occurs on a computer). In some embodiments, the method further comprises administering an additional amount of the Klotho polypeptide to the animal if the quantity or activity is below the control value. In some embodiments, the animal is a human. In some embodiments, a second sample is obtained from the animal and quantity of the polypeptide is compared between a first sample and a second sample.

Also provided is a method of assessing an animal as a candidate for improved cognition treatment. In some embodiments, the method comprises obtaining a sample from the animal and measuring a quantity or activity of any one or more a polypeptide of Table 1 or Table 2; comparing the quantity or activity to a control value; and then administering an effective dose of a Klotho polypeptide or a protein comprising a polypeptide of Table 1 or Table 2 or a functional fragment or variant thereof to the animal to improve cognition in the animal. In some embodiments, the sample comprises platelets. In some embodiments, the obtaining comprises purifying platelets from blood from the animal. In some embodiments, the method further comprises after the administering, obtaining a second sample from the animal and measuring the quantity or activity of any one or more the polypeptide of Table 1 or Table 2; and comparing the quantity or activity from the second sample to a control value or to the quantity from the first sample. In some embodiments, the animal is a human.

### BRIEF DESCRIPTION OF THE DRAWINGS

FIG. 1A-C: (1A) Paradigm for plasma proteomics profiling. Mice were injected with klotho, allowed to explore a small Y-maze for 10 minutes, and then their plasma was immediately harvested. (1B) Plasma proteomics identified PF4 as highest expressed klotho-induced protein (6.5 fold increase, FDR q-value=0.002). (1C) Pathway analysis predicts klotho activates platelets and their functions.

FIG. 2A-B: (2A) Paradigm of Veh or PF4 Trt in aging mice followed by cognitive testing (n=8 mice/group, age 18-21 mos). (2B) PF4 treatment given one hour before training and then 1 h before testing increased memory of aging mice, measured by time spent exploring the novel compared to familiar arms of the Two Trials Y-maze over several minutes (Two-way repeated measures ANOVA, *p<0.05). Data are mean±SEM.

FIG. 3A-C: (3A) Paradigm for measuring platelet activation. Mice (age 5 months; n=8-9 mice per group) were treated with either Veh or Klotho (s.c., 10 μg/kg) followed by platelet isolation from whole blood and then platelet activation analysis by fluorescence activated single cell sorting (FACS) sorting with markers CD61 and CD62P. (3B) Flow cytometry plots from FACS sorting show platelet populations. The upper graphs show density plots of the platelets, gated by SSC (for granularity) and CD61-positivity which both identify platelets from other blood cells. The lower graphs show dot plots of the percentage activated (CD61 and CD62P-positive) and resting (CD61-positive only) platelets. (3C) Quantification of activated platelets in young mice following treatment with Veh or Klotho.

### DEFINITIONS

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

The terms “klotho” or “klotho polypeptide” refer to soluble klotho polypeptide, or functional variants and fragments thereof, including those described herein, unless otherwise stated. Soluble klotho is any form of klotho that circulates in fluid (e.g., serum, cerebrospinal fluid, etc.), and that does not include a transmembrane or intracellular component. Klotho can be cleaved from its transmembrane form and released into fluid, or otherwise secreted or shed from a cell. Klotho RNA can also be alternatively spliced and directly secreted into the surrounding fluid (i.e., without forming a transmembrane protein). Both protein forms are encompassed in the terms soluble klotho polypeptide, klotho polypeptide, and klotho.

As used herein, the terms “systemic” or “peripheral” refer to administration by a route that does not involve direct injection (or other administration) into the cerebrospinal fluid (CSF) or central nervous system (CNS). That is, systemic and peripheral administration encompasses administration to the “blood” side of the blood-brain barrier. Examples of systemic and peripheral routes include oral and mucosal, intravenous, intraperitoneal, intramuscular, and subcutaneous injection, and intravenous drip.

The terms “cognition,” “cognitive ability,” “cognitive function,” and like terms refer to a collection of mental tasks and functions, including but not limited to: memory (e.g., semantic, episodic, procedural, priming, or working); orientation; language; problem solving; visual perception, construction, and integration; planning; organizational skills; selective attention; inhibitory control; and ability to mentally manipulate information.

The terms “improved cognition,” “increased cognitive ability,” “improved cognitive function,” and like terms refer to an improvement in cognition under a given condition (e.g. treatment with klotho) compared to cognition absent the condition (e.g., absent treatment with klotho). For an individual experiencing cognitive decline, an improvement in cognition might be a reduction in the rate of cognitive decline (i.e., an improvement compared to the absence of treatment), but not an actual improvement in cognitive ability. An increase in cognitive ability can also be an increase in brain activity in a specified area, e.g., as determined by MRI, or an inhibition of brain activity that results in better overall brain function. An increase in cognitive ability can also be improvement in a cognitive performance test as described in more detail herein. An improvement or increase in cognitive ability can be in any one cognitive aspect or function, or any combination of individual cognitive functions.

An individual in need of improved cognitive function refers to individuals with age-related cognitive decline; a neurodegenerative disease; a mental or mood disorder; traumatic brain injury; developmental delay; genetic disorder resulting in reduced cognitive ability; brain injury due to stroke, brain cancer, MS, epilepsy, radiation or chemotherapy; etc. An individual in need of improved cognitive function can also include individuals that desire increased mental function to fight the effects of stress, sleep deprivation, jet lag, or pain, or to heighten ability for a particular task.

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following amino acids are typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “identical” or “percent identity,” in the context of two or more nucleic acids, or two or more polypeptides, refer to two or more sequences or subsequences that are the same or have a specified percentage of nucleotides, or amino acids, that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” This definition also refers to, or may be applied to, the compliment of a nucleotide test sequence. The definition also includes sequences that have deletions and/or additions, as well as those that have substitutions. As described below, the algorithms can account for gaps and the like. Typically, identity exists over a region comprising an antibody epitope, or a sequence that is at least about 25 amino acids or nucleotides in length, or over a region that is 50-100 amino acids or nucleotides in length, or over the entire length of the reference sequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a protein or nucleic acid indicates that the protein or nucleic acid comprises two or more subsequences that are not found in the same relationship to each other in nature. For instance, the protein or nucleic acid is typically recombinantly produced, having two or more sequences from unrelated genes arranged to make a new functional nucleic acid, e.g., a promoter from one source and a coding region from another source, or functional chimeric protein.

The terms “effective amount,” “effective dose,” “therapeutically effective amount,” etc. refer to that amount of the therapeutic agent sufficient to ameliorate a disorder. For example, for the given parameter, a therapeutically effective amount will show an increase or decrease of therapeutic effect at least 1%, 2%, 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “pharmaceutically acceptable” is used synonymously with physiologically acceptable and pharmacologically acceptable. A pharmaceutical composition will generally comprise agents for buffering and preservation in storage, and can include buffers and carriers for appropriate delivery, depending on the route of administration.

The terms “dose” and “dosage” are used interchangeably herein. A dose refers to the amount of active ingredient given to an individual at each administration. For example the dose can refer to the amount of Klotho polypeptide. The dose will vary depending on a number of factors, including frequency of administration; size and tolerance of the individual; type and severity of the condition; risk of side effects; and the route of administration. One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as dogs, horses, pigs, mice, rats, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc.

The terms “specific for,” “specifically binds,” and like terms refer to a molecule (e.g., antibody or antibody fragment) that binds to a target with at least 2-fold greater affinity than non-target compounds, e.g., at least any of 4-fold, 5-fold, 6-fold, 7-fold, 8-fold, 9-fold, 10-fold, 20-fold, 25-fold, 50-fold, or 100-fold greater affinity. Specificity can be determined using standard methods, e.g., solid-phase ELISA immunoassays (see, e.g., Harlow & Lane, Using Antibodies, A Laboratory Manual (1998) for a description of immunoassay formats and conditions that can be used to determine specific immunoreactivity).

### DETAILED DESCRIPTION OF THE INVENTION

The Klotho protein's role in cognition and other biological processes has been established, but in the past Klotho's mechanism of action and signal transduction pathway has not been known. As a result, while Klotho could be administered in clinical, preclinical and experimental situations, it was not possible to measure whether Klotho induced a biological activity other than by measuring the desired result (e.g., improved cognition). As described herein, Klotho activates platelets and thus proteins indicating activation of platelets can be measured in an animal (e.g., a human) to measure Klotho activity in an animal either before administration of Klotho (e.g., to identify how likely an animal is to be most responsive to Klotho) or after administration of Klotho to measure Klotho's effect on downstream intermediates.

A number of proteins have been discovered or determined whose quantity (e.g., expression or steady—state amount) or activity increases with administration of activate Klotho. Table 1 lists proteins identified as enriched in response to Klotho and thus can be used as described herein to measure Klotho activity as described herein. Table 2 lists proteins involved in platelet activation and thus in view of Klotho's effect on platelet activation and platelet factors, can also be used to measure Klotho activity.

| | | Ratio | |
|---|---|---|---|
| | | (KI/ | |
| Name | GenBank ID/Uniprot ID | Veh) | Gen. Functions |
| Platelet factor | 5196 | 6.55 | PF4 is a small cytokine |
| 4 (PF4, CXCL4) | (human) | | released from alpha- |
| | EAEEDGDLQCLCVKTTSQV | | granules of activated |
| | RPRHITSLEV IKAGPHCPTA | | platelets during |
| | QLIATLKNGR KICLDLQAPL | | platelet aggregation, |
| | YKKIIKKLLES | | exercise, and poten- |
| | (SEQ ID NO: 2)/Q9Z126 | | tially other situa- |
| | (mouse) | | tions. It is involved |
| | | | in blood coagulation |
| | | | and neurogenesis and |
| | | | is related to anti- |
| | | | cancer actions (Leiter |
| | | | O, Seidemann S, Overall |
| | | | R W, et al. Stem Cell |
| | | | Reports. 2019; 12(4): |
| | | | 667-679). Its receptor |
| | | | is CXCR (de Jong E K, |
| | | | de Haas A H, Brouwer N, |
| | | | et al. J Neurochem. |
| | | | 2008; 105(5):1726- |
| | | | 1736). It may act |
| | | | more potently when |
| | | | combined with IL-8 |
| | | | (Nesmelova I V, Sham Y, |
| | | | Dudek A Z, et al. J |
| | | | Biol Chem. 2005; |
| | | | 280(6):4948-4958) (or |
| | | | potentially with other |
| | | | blood factors or with |
| | | | klotho, being tested). |
| Thrombospondin- | 7057 | 4.04 | THB1 or TSP1 is an |
| 1 (THBS1, TSP1) | (human) | | adhesive glycoprotein |
| | MGLAWGLGVLFLMHVCGTNRIPESGGD | | involved in cell-cell |
| | NSVFDIFELTGAARKGSGRRLVKGPDPS | | and cell-matrix in- |
| | SPAFRIEDANLIPPVPDDKFQDLVDAVRA | | teractions. It plays |
| | EKGFLLLASLRQMKKTRGTLLALERKDH | | roles in platelet |
| | SGQVFSVVSNGKAGTLDLSLTVQGKQH | | aggregation, angio- |
| | VVSVEEALLATGQWKSITLFVQEDRAQL | | genesis, tumorigenesis |
| | YIDCEKMENAELDVPIQSVFTRDLASIAR | | (Isenberg J S, Romeo |
| | LRIAKGGVNDNFQGVLQNVRFVFGTTPE | | M J, Yu C, et al. |
| | DILRNKGCSSSTSVLLTLDNNVVNGSSP | | Blood. 2008; 111(2): |
| | AIRTNYIGHKTKDLQAICGISCDELSSMVL | | 613-623; Sheibani N, |
| | ELRGLRTIVTTLQDSIRKVTEENKELANE | | Frazier W A. Proc |
| | LRRPPLCYHNGVQYRNNEEWTVDSCTE | | Natl Acad Sci USA. |
| | CHCQNSVTICKKVSCPIMPCSNATVPDG | | 1995; 92(15):6788- |
| | ECCPRCWPSDSADDGWSPWSEWTSC | | 6792), and facilitates |
| | STSCGNGIQQRGRSCDSLNNRCEGSSV | | synapse formation in |
| | QTRTCHIQECDKRFKQDGGWSHWSPW | | hippocampal |
| | SSCSVTCGDGVITRIRLCNSPSPQMNGK | | neurons through neuro- |
| | PCEGEARETKACKKDACPINGGWGPWS | | ligin-1 (Xu J, Xiao N, |
| | PWDICSVTCGGGVQKRSRLCNNPTPQF | | Xia J. Nat Neurosci. |
| | GGKDCVGDVTENQICNKQDCPIDGCLS | | 2010; 13(1):22-24). |
| | NPCFAGVKCTSYPDGSWKCGACPPGY | | |
| | SGNGIQCTDVDECKEVPDACFNHNGEH | | |
| | RCENTDPGYNCLPCPPRFTGSQPFGQG | | |
| | VEHATANKQVCKPRNPCTDGTHDCNKN | | |
| | AKCNYLGHYSDPMYRCECKPGYAGNGII | | |
| | CGEDTDLDGWPNENLVCVANATYHCKK | | |
| | DNCPNLPNSGQEDYDKDGIGDACDDDD | | |
| | DNDKIPDDRDNCPFHYNPAQYDYDRDD | | |
| | VGDRCDNCPYNHNPDQADTDNNGEGD | | |
| | ACAADIDGDGILNERDNCQYVYNVDQR | | |
| | DTDMDGVGDQCDNCPLEHNPDQLDSD | | |
| | SDRIGDTCDNNQDIDEDGHQNNLDNCP | | |
| | YVPNANQADHDKDGKGDACDHDDDND | | |
| | GIPDDKDNCRLVPNPDQKDSDGDGRGD | | |
| | ACKDDFDHDSVPDIDDICPENVDISETDF | | |
| | RRFQMIPLDPKGTSQNDPNWVVRHQGK | | |
| | ELVQTVNCDPGLAVGYDEFNAVDFSGT | | |
| | FFINTERDDDYAGFVFGYQSSSRFYVVM | | |
| | WKQVTQSYWDTNPTRAQGYSGLSVKV | | |
| | VNSTTGPGEHLRNALWHTGNTPGQVRT | | |
| | LWHDPRHIGWKDFTAYRWRLSHRPKTG | | |
| | FIRVVMYEGKKIMADSGPIYDKTYAGGR | | |
| | LGLFVFSQEMVFFSDLKYECRDP | | |
| | (SEQ ID NO: 3)/P35441 | | |
| | (mouse) | | |
| Fermitin family | 83706 | 3.95 | FERT3 is a key molecule |
| homolog 3 | (human) | | for organization of fo- |
| (FERMT3) | MAGMKTASGDYIDSSWELRVFVGEEDP | | cal adhesions that con- |
| | EAESVTLRVTGESHIGGVLLKIVEQINRK | | nect cell-extracellular |
| | QDWSDHAIWWEQKRQWLLQTHWTLDK | | matrix junctions; it |
| | YGILADARLFFGPQHRPVILRLPNRRALR | | also controls cell-cell |
| | LRASFSQPLFQAVAAICRLLSIRHPEELS | | contacts and nucleus |
| | LLRAPEKKEKKKKEKEPEEELYDLSKVVL | | function (Li H, Deng Y, |
| | AGGVAPALFRGMPAHFSDSAQTEACYH | | Sun K, et al. Proc Natl |
| | MLSRPQPPPDPLLLQRLPRPSSLSDKTQ | | Acad Sci USA. 2017; |
| | LHSRWLDSSRCLMQQGIKAGDALWLRF | | 114(35):9349-9354). |
| | KYYSFFDLDPKTDPVRLTQLYEQARWDL | | |
| | LLEEIDCTEEEMMVFAALQYHINKLSQSG | | |
| | EVGEPAGTDPGLDDLDVALSNLEVKLEG | | |
| | SAPTDVLDSLTTIPELKDHLRIFRIPRRPR | | |
| | KLTLKGYRQHWVVFKETTLSYYKSQDEA | | |
| | PGDPIQQLNLKGCEVVPDVNVSGQKFCI | | |
| | KLLVPSPEGMSEIYLRCQDEQQYARWM | | |
| | AGCRLASKGRTMADSSYTSEVQAILAFL | | |
| | SLQRTGSGGPGNHPHGPDASAEGLNPY | | |
| | GLVAPRFQRKFKAKQLTPRILEAHQNVA | | |
| | QLSLAEAQLRFIQAWQSLPDFGISYVMV | | |
| | RFKGSRKDEILGIANNRLIRIDLAVGDVVK | | |
| | TWRFSNMRQWNVNWDIRQVAIEFDEHI | | |
| | NVAFSCVSASCRIVHEYIGGYIFLSTRER | | |
| | ARGEELDEDLFLQLTGGHEAF | | |
| | (SEQ ID NO: 4)/Q8K1B8 | | |
| | (mouse) | | |
| Talin-1 (TLN1) | 7094 | 2.77 | TLN1 is ubiquitously |
| | (human) | | expressed and mediates |
| | MVALSLKISIGNVVKTMQFEPSTMVYDA | | cell-cell adhesion by |
| | CRIIRERIPEAPAGPPSDFGLFLSDDDPK | | linking integrins to |
| | KGIWLEAGKALDYYMLRNGDTMEYRKK | | the actin cytoskeleton; |
| | QRPLKIRMLDGTVKTIMVDDSKTVTDML | | it also participates in |
| | MTICARIGITNHDEYSLVRELMEEKKEEIT | | the activation of in- |
| | GTLRKDKTLLRDEKKMEKLKQKLHTDDE | | tegrins (Manso A M, |
| | LNWLDHGRTLREQGVEEHETLLLRRKFF | | Okada H, Sakamoto F |
| | YSDQNVDSRDPVQLNLLYVQARDDILNG | | M, et al. Proc Natl |
| | SHPVSFDKACEFAGFQCQIQFGPHNEQ | | Acad Sci USA. 2017; |
| | KHKAGFLDLKDFLPKEYVKQKGERKIFQ | | 114(30):E6250-E6259). |
| | AHKNCGQMSEIEAKVRYVKLARSLKTYG | | |
| | VSFFLVKEKMKGKNKLVPRLLGITKECV | | |
| | MRVDEKTKEVIQEWNLTNIKRWAASPKS | | |
| | FTLDFGDYQDGYYSVQTTEGEQIAQLIA | | |
| | GYIDIILKKKKSKDHFGLEGDEESTMLED | | |
| | SVSPKKSTVLQQQYNRVGKVEHGSVAL | | |
| | PAIMRSGASGPENFQVGSMPPAQQQIT | | |
| | SGQMHRGHMPPLTSAQQALTGTINSSM | | |
| | QAVQAAQATLDDFDTLPPLGQDAASKA | | |
| | WRKNKMDESKHEIHSQVDAITAGTASVV | | |
| | NLTAGDPAETDYTAVGCAVTTISSNLTE | | |
| | MSRGVKLLAALLEDEGGSGRPLLQAAK | | |
| | GLAGAVSELLRSAQPASAEPRQNLLQAA | | |
| | GNVGQASGELLQQIGESDTDPHFQDAL | | |
| | MQLAKAVASAAAALVLKAKSVAQRTEDS | | |
| | GLQTQVIAAATQCALSTSQLVACTKVVA | | |
| | PTISSPVCQEQLVEAGRLVAKAVEGCVS | | |
| | ASQAATEDGQLLRGVGAAATAVTQALN | | |
| | ELLQHVKAHATGAGPAGRYDQATDTILT | | |
| | VTENIFSSMGDAGEMVRQARILAQATSD | | |
| | LVNAIKADAEGESDLENSRKLLSAAKILA | | |
| | DATAKMVEAAKGAAAHPDSEEQQQRLR | | |
| | EAAEGLRMATNAAAQNAIKKKLVQRLEH | | |
| | AAKQAAASATQTIAAAQHAASTPKASAG | | |
| | PQPLLVQSCKAVAEQIPLLVQGVRGSQA | | |
| | QPDSPSAQLALIAASQSFLQPGGKMVAA | | |
| | AKASVPTIQDQASAMQLSQCAKNLGTAL | | |
| | AELRTAAQKAQEACGPLEMDSALSVVQ | | |
| | NLEKDLQEVKAAARDGKLKPLPGETMEK | | |
| | CTQDLGNSTKAVSSAIAQLLGEVAQGNE | | |
| | NYAGIAARDVAGGLRSLAQAARGVAALT | | |
| | SDPAVQAIVLDTASDVLDKASSLIEEAKK | | |
| | AAGHPGDPESQQRLAQVAKAVTQALNR | | |
| | CVSCLPGQRDVDNALRAVGDASKRLLS | | |
| | DSLPPSTGTFQEAQSRLNEAAAGLNQA | | |
| | ATELVQASRGTPQDLARASGRFGQDFS | | |
| | TFLEAGVEMAGQAPSQEDRAQVVSNLK | | |
| | GISMSSSKLLLAAKALSTDPAAPNLKSQL | | |
| | AAAARAVTDSINQLITMCTQQAPGQKEC | | |
| | DNALRELETVRELLENPVQPINDMSYFG | | |
| | CLDSVMENSKVLGEAMTGISQNAKNGN | | |
| | LPEFGDAISTASKALCGFTEAAAQAAYLV | | |
| | GVSDPNSQAGQQGLVEPTQFARANQAI | | |
| | QMACQSLGEPGCTQAQVLSAATIVAKHT | | |
| | SALCNSCRLASARTTNPTAKRQFVQSAK | | |
| | EVANSTANLVKTIKALDGAFTEENRAQC | | |
| | RAATAPLLEAVDNLSAFASNPEFSSIPAQ | | |
| | ISPEGRAAMEPIVISAKTMLESAGGLIQT | | |
| | ARALAVNPRDPPSWSVLAGHSRTVSDSI | | |
| | KKLITSMRDKAPGQLECETAIAALNSCLR | | |
| | DLDQASLAAVSQQLAPREGISQEALHTQ | | |
| | MLTAVQEISHLIEPLANAARAEASQLGHK | | |
| | VSQMAQYFEPLTLAAVGAASKTLSHPQ | | |
| | QMALLDQTKTLAESALQLLYTAKEAGGN | | |
| | PKQAAHTQEALEEAVQMMTEAVEDLTT | | |
| | TLNEAASAAGVVGGMVDSITQAINQLDE | | |
| | GPMGEPEGSFVDYQTTMVRTAKAIAVTV | | |
| | QEMVTKSNTSPEELGPLANQLTSDYGRL | | |
| | ASEAKPAAVAAENEEIGSHIKHRVQELG | | |
| | HGCAALVTKAGALQCSPSDAYTKKELIE | | |
| | CARRVSEKVSHVLAALQAGNRGTQACIT | | |
| | AASAVSGIIADLDTTIMFATAGTLNREGT | | |
| | ETFADHREGILKTAKVLVEDTKVLVQNAA | | |
| | GSQEKLAQAAQSSVATITRLADVVKLGA | | |
| | ASLGAEDPETQVVLINAVKDVAKALGDLI | | |
| | SATKAAAGKVGDDPAVWQLKNSAKVMV | | |
| | TNVTSLLKTVKAVEDEATKGTRALEATTE | | |
| | HIRQELAVFCSPEPPAKTSTPEDFIRMTK | | |
| | GITMATAKAVAAGNSCRQEDVIATANLS | | |
| | RRAIADMLRACKEAAYHPEVAPDVRLRA | | |
| | LHYGRECANGYLELLDHVLLTLQKPSPE | | |
| | LKQQLTGHSKRVAGSVTELIQAAEAMKG | | |
| | TEWVDPEDPTVIAENELLGAAAAIEAAAK | | |
| | KLEQLKPRAKPKEADESLNFEEQILEAAK | | |
| | SIAAATSALVKAASAAQRELVAQGKVGAI | | |
| | PANALDDGQWSQGLISAARMVAAATNN | | |
| | LCEAANAAVQGHASQEKLISSAKQVAAS | | |
| | TAQLLVACKVKADQDSEAMKRLQAAGN | | |
| | AVKRASDNLVKAAQKAAAFEEQENETVV | | |
| | VKEKMVGGIAQIIAAQEEMLRKERELEEA | | |
| | RKKLAQIRQQQYKFLPSELRDEH | | |
| | (SEQ ID NO: 5)/P26039 | | |
| | (mouse) | | |
| Creatine | 1158 | 2.41 | CKM catalyzes the |
| kinase | (human) | | transfer of phosphate |
| M-type | MPFGNTHNKFKLNYKPEEEYPDLSKHN | | between ATP and |
| (CKM) | NHMAKVLTLELYKKLRDKETPSGFTVDD | | creatine; it also |
| | VIQTGVDNPGHPFIMTVGCVAGDEESYE | | catalyzes the transfer |
| | VFKELFDPIISDRHGGYKPTDKHKTDLNH | | of phosphate between |
| | ENLKGGDDLDPNYVLSSRVRTGRSIKGY | | phospho-creatine and |
| | TLPPHCSRGERRAVEKLSVEALNSLTGE | | ADP (Schafer B, |
| | FKGKYYPLKSMTEKEQQQLIDDHFLFDK | | Perriard J C, |
| | PVSPLLLASGMARDWPDARGIWHNDNK | | Eppenberger H M. |
| | SFLVWVNEEDHLRVISMEKGGNMKEVF | | Basic Res Cardiol. |
| | RRFCVGLQKIEEIFKKAGHPFMWNQHLG | | 1985; 80 Suppl 2: |
| | YVLTCPSNLGTGLRGGVHVKLAHLSKHP | | 129-133). |
| | KFEEILTRLRLQKRGTGGVDTAAVGSVF | | |
| | DVSNADRLGSSEVEQVQLVVDGVKLMV | | |
| | EMEKKLEKGQSIDDMIPAQK | | |
| | (SEQ ID NO: 6)/P07310 | | |
| | (mouse) | | |
| Glyceraldehyde- | 2597 | 2.05 | GAPDH is involved in |
| 3-phosphate | (human) | | catalyzing the sixth |
| dehydrogenase | MGKVKVGVNGFGRIGRLVTRAAFNSGK | | step of glycolysis; it |
| (GAPDH) | VDIVAINDPFIDLNYMVYMFQYDSTHGKF | | breaks down glucose |
| | HGTVKAENGKLVINGNPITIFQERDPSKI | | for energy and carbon |
| | KWGDAGAEYVVESTGVFTTMEKAGAHL | | molecules (Yang J S, |
| | QGGAKRVIISAPSADAPMFVMGVNHEKY | | Hsu J W, Park S Y, et |
| | DNSLKIISNASCTTNCLAPLAKVIHDNFGI | | al. Nature. 2018; 561 |
| | VEGLMTTVHAITATQKTVDGPSGKLWRD | | (7722):263-267). |
| | GRGALQNIIPASTGAAKAVGKVIPELNGK | | |
| | LTGMAFRVPTANVSVVDLTCRLEKPAKY | | |
| | DDIKKVVKQASEGPLKGILGYTEHQVVS | | |
| | SDFNSDTHSSTFDAGAGIALNDHFVKLIS | | |
| | WYDNEFGYSNRVVDLMAHMASKE | | |
| | (SEQ ID NO: 7)/P16858 | | |
| | (mouse) | | |
| Elongation | 1915 | 1.79 | EEF1A1 enzymatically |
| factor | (human) | | delivers aminoacyl |
| 1-alpha 1 | MGKEKTHINIVVIGHVDSGKSTTTGHLIY | | tRNAs to the ribosome |
| (EEF1A1) | KCGGIDKRTIEKFEKEAAEMGKGSFKYA | | (Vera M, Pani B, |
| | WVLDKLKAERERGITIDISLWKFETSKYY | | Griffiths L A, et al. |
| | VTIIDAPGHRDFIKNMITGTSQADCAVLIV | | Elife. 2014; 3:e03164). |
| | AAGVGEFEAGISKNGQTREHALLAYTLG | | |
| | VKQLIVGVNKMDSTEPPYSQKRYEEIVK | | |
| | EVSTYIKKIGYNPDTVAFVPISGWNGDN | | |
| | MLEPSANMPWFKGWKVTRKDGNASGT | | |
| | TLLEALDCILPPTRPTDKPLRLPLQDVYKI | | |
| | GGIGTVPVGRVETGVLKPGMVVTFAPVN | | |
| | VTTEVKSVEMHHEALSEALPGDNVGFN | | |
| | VKNVSVKDVRRGNVAGDSKNDPPMEAA | | |
| | GFTAQVIILNHPGQISAGYAPVLDCHTAH | | |
| | IACKFAELKEKIDRRSGKKLEDGPKFLKS | | |
| | GDAAIVDMVPGKPMCVESFSDYPPLGR | | |
| | FAVRDMRQTVAVGVIKAVDKKAAGAGK | | |
| | VTKSAQKAQKAK | | |
| | (SEQ ID NO: 8)/P10126 | | |
| | (mouse) | | |
| Ig gamma-1 | 3500 | 1.43 | IGHG1 is a constant |
| chain | (human) | | region of immunoglo- |
| C region | ASTKGPSVFPLAPSSKSTSGGTAALGCL | | bulin heavy chains and |
| secreted | VKDYFPEPVTVSWNSGALTSGVHTFPA | | is involved in the |
| form (IGHG1) | VLQSSGLYSLSSVVTVPSSSLGTQTYICN | | growth of cancers |
| | VNHKPSNTKVDKKVEPKSCDKTHTCPP | | (Chu J, Li Y, Deng Z, |
| | CPAPELLGGPSVFLFPPKPKDTLMISRTP | | et al. IGHG1 Biomed |
| | EVTCVVVDVSHEDPEVKFNWYVDGVEV | | Res Int. 2019; 2019: |
| | HNAKTKPREEQYNSTYRVVSVLTVLHQD | | 7201562). |
| | WLNGKEYKCKVSNKALPAPIEKTISKAK | | |
| | GQPREPQVYTLPPSRDELTKNQVSLTCL | | |
| | VKGFYPSDIAVEWESNGQPENNYKTTP | | |
| | PVLDSDGSFFLYSKLTVDKSRWQQGNV | | |
| | FSCSVMHEALHNHYTQKSLSLSPGK | | |
| | (SEQ ID NO: 9)/P01868 | | |
| | (mouse) | | |
| Tubulin | 7846 | 1.36 | TUBA1A is part of the |
| alpha-1A | (human) | | formation of micro- |
| chain | MRECISIHVGQAGVQIGNACWELYCLEH | | tubules structural |
| (TUBA1A) | GIQPDGQMPSDKTIGGGDDSFNTFFSET | | proteins that partici- |
| | GAGKHVPRAVFVDLEPTVIDEVRTGTYR | | pate in cytoskeletal |
| | QLFHPEQLITGKEDAANNYARGHYTIGK | | structure. Importantly, |
| | EIIDLVLDRIRKLADQCTGLQGFLVFHSF | | it functions in the |
| | GGGTGSGFTSLLMERLSVDYGKKSKLE | | adult hippocampal |
| | FSIYPAPQVSTAVVEPYNSILTTHTTLEH | | neurogenesis and |
| | SDCAFMVDNEAIYDICRRNLDIERPTYTN | | formation of dentate |
| | LNRLIGQIVSSITASLRFDGALNVDLTEFQ | | gyrus (Keays D A, |
| | TNLVPYPRIHFPLATYAPVISAEKAYHEQ | | Cleak J, Huang G J, |
| | LSVAEITNACFEPANQMVKCDPRHGKY | | et al. Dev Neurosci. |
| | MACCLLYRGDVVPKDVNAAIATIKTKRTI | | 2010; 32(4):268-277). |
| | QFVDWCPTGFKVGINYQPPTVVPGGDL | | |
| | AKVQRAVCMLSNTTAIAEAWARLDHKFD | | |
| | LMYAKRAFVHWYVGEGMEEGEFSEARE | | |
| | DMAALEKDYEEVGVDSVEGEGEEEGEE | | |
| | Y | | |
| | (SEQ ID NO: 10)/P68369 | | |
| | (mouse) | | |
| Heat shock | P11142(human) | 1.33 | HSPA8 facilitates |
| cognate 71 | MSKGPAVGIDLGTTYSCVGVFQHGKVEII | | proper folding of |
| kDa protein | ANDQGNRTTPSYVAFTDTERLIGDAAKN | | newly translated and |
| (HSPA8) | QVAMNPTNTVFDAKRLIGRRFDDAVVQS | | misfolded proteins; it |
| | DMKHWPFMVVNDAGRPKVQVEYKGET | | also stabilizes or |
| | KSFYPEEVSSMVLTKMKEIAEAYLGKTV | | degrades mutant pro- |
| | TNAVVTVPAYFNDSQRQATKDAGTIAGL | | teins, it fundamentally |
| | NVLRIINEPTAAAIAYGLDKKVGAERNVLI | | functions in various |
| | FDLGGGTFDVSILTIEDGIFEVKSTAGDT | | biological processes |
| | HLGGEDFDNRMVNHFIAEFKRKHKKDIS | | including signal trans- |
| | ENKRAVRRLRTACERAKRTLSSSTQASI | | duction, protein homeo- |
| | EIDSLYEGIDFYTSITRARFEELNADLFRG | | stasis, and cell |
| | TLDPVEKALRDAKLDKSQIHDIVLVGGST | | growth/differentiation |
| | RIPKIQKLLQDFFNGKELNKSINPDEAVA | | (Wang F, Bonam S R, |
| | YGAAVQAAILSGDKSENVQDLLLLDVTPL | | Schall N, et al. Sci |
| | SLGIETAGGVMTVLIKRNTTIPTKQTQTF | | Rep. 2018; 8(1): |
| | TTYSDNQPGVLIQVYEGERAMTKDNNLL | | 16820). |
| | GKFELTGIPPAPRGVPQIEVTFDIDANGIL | | |
| | NVSAVDKSTGKENKITITNDKGRLSKEDI | | |
| | ERMVQEAEKYKAEDEKQRDKVSSKNSL | | |
| | ESYAFNMKATVEDEKLQGKINDEDKQKI | | |
| | LDKCNEIINWLDKNQTAEKEEFEHQQKE | | |
| | LEKVCNPIITKLYQSAGGMPGGMPGGFP | | |
| | GGGAPPSGGASSGPTIEEVD | | |
| | (SEQ ID NO: 11)/P63017 | | |
| | (mouse) | | |
| Catalase (CAT) | 3312 | 1.27 | CAT catalyzes the de- |
| | (human) | | composition of |
| | ADSRDPASDQMQHWKEQRAAQKADVL | | hydrogen peroxide to |
| | TTGAGNPVGDKLNVITVGPRGPLLVQDV | | water and oxygen (Peng |
| | VFTDEMAHFDRERIPERVVHAKGAGAF | | J, Stevenson F F, |
| | GYFEVTHDITKYSKAKVFEHIGKKTPIAV | | Doctrow S R, Andersen |
| | RFSTVAGESGSADTVRDPRGFAVKFYT | | J K. J Biol Chem. 2005; |
| | EDGNWDLVGNNTPIFFIRDPILFPSFIHS | | 280(32):29194-29198) |
| | QKRNPQTHLKDPDMVWDFWSLRPESL | | |
| | HQVSFLFSDRGIPDGHRHMNGYGSHTF | | |
| | KLVNANGEAVYCKFHYKTDQGIKNLSVE | | |
| | DAARLSQEDPDYGIRDLFNAIATGKYPS | | |
| | WTFYIQVMTFNQAETFPFNPFDLTKVWP | | |
| | HKDYPLIPVGKLVLNRNPVNYFAEVEQIA | | |
| | FDPSNMPPGIEASPDKMLQGRLFAYPDT | | |
| | HRHRLGPNYLHIPVNCPYRARVANYQR | | |
| | DGPMCMQDNQGGAPNYYPNSFGAPEQ | | |
| | QPSALEHSIQYSGEVRRFNTANDDNVTQ | | |
| | VRAFYVNVLNEEQRKRLCENIAGHLKDA | | |
| | QIFIQKKAVKNFTEVHPDYGSHIQALLDK | | |
| | YNAEKPKNAIHTFVQSGSHLAAREKANL | | |
| | (SEQ ID NO: 12)/P24270 | | |
| | (mouse) | | |
| Actin, | 60; 71 | 1.23 | ACTG1 polymerizes to |
| cytoplasmic | (human) | | make filaments by |
| 1; Actin, | MDDDIAALVVDNGSGMCKAGFAGDDAP | | forming cross-linked |
| cytoplasmic | RAVFPSIVGRPRHQGVMVGMGQKDSYV | | networks in the |
| 2 (ACTB; | GDEAQSKRGILTLKYPIEHGIVTNWDDM | | cytoplasm of cells |
| ACTG1) | EKIWHHTFYNELRVAPEEHPVLLTEAPL | | to facilitate motility |
| | NPKANREKMTQIMFETFNTPAMYVAIQA | | and contraction |
| | VLSLYASGRTTGIVMDSGDGVTHTVPIY | | (Hsueh Y P. Commun |
| | EGYALPHAILRLDLAGRDLTDYLMKILTE | | Integr Biol. 2012; |
| | RGYSFTTTAEREIVRDIKEKLCYVALDFE | | 5(4):334-336.) |
| | QEMATAASSSSLEKSYELPDGQVITIGN | | |
| | ERFRCPEALFQPSFLGMESCGIHETTFN | | |
| | SIMKCDVDIRKDLYANTVLSGGTTMYPGI | | |
| | ADRMQKEITALAPSTMKIKIIAPPERKYSV | | |
| | WIGGSILASLSTFQQMWISKQEYDESGP | | |
| | SIVHRKCF | | |
| | (SEQ ID NO: 13)/P60710; | | |
| | P63260 | | |
| | (mouse) | | |
| Hemoglobin | 15129 | 1.21 | HBB-B1 is the most |
| subunit | (mouse) | | common form of hemo- |
| beta-1 | MVHLTPEEKSAVTALWGKVNVDEVGGE | | globin in adult |
| (HBB-B1) | ALGRLLVVYPWTQRFFESFGDLSTPDAV | | humans; it is in- |
| | MGNPKVKAHGKKVLGAFSDGLAHLDNL | | volved in some |
| | KGTFATLSELHCDKLHVDPENFRLLGNV | | genetic disorders |
| | LVCVLAHHFGKEFTPPVQAAYQKVVAGV | | such as sickle-cell |
| | ANALAHKYH | | and beta thalassemia |
| | (SEQ ID NO: 14)/P02088 | | (Chang C K, Simplaceanu |
| | (mouse) | | V, Ho C. Biochemistry. |
| | | | 2002; 41(17):5644-5655). |
| Phospholipid | 5360 | 1.17 | PLTP transfers phospho- |
| transfer | (human) | | lipids from tri- |
| protein | MALFGALFLALLAGAHAEFPGCKIRVTSK | | glyceride-rich lipo- |
| (PLTP) | ALELVKQEGLRFLEQELETITIPDLRGKE | | proteins to high |
| | GHFYYNISEVKVTELQLTSSELDFQPQQ | | density lipoprotein |
| | ELMLQITNASLGLRFRRQLLYWFFYDGG | | (HDL) and is involved |
| | YINASAEGVSIRTGLELSRDPAGRMKVS | | in cholesterol |
| | NVSCQASVSRMHAAFGGTFKKVYDFLS | | metabolism (Desrumaux |
| | TFITSGMRFLLNQQICPVLYHAGTVLLNS | | C, Risold P Y, |
| | LLDTVPVRSSVDELVGIDYSLMKDPVAS | | Schroeder H, et al. |
| | TSNLDMDFRGAFFPLTERNWSLPNRAV | | FASEB J. 2005; |
| | EPQLQEEERMVYVAFSEFFFDSAMESY | | 19(2):296-297). |
| | FRAGALQLLLVGDKVPHDLDMLLRATYF | | |
| | GSIVLLSPAVIDSPLKLELRVLAPPRCTIK | | |
| | PSGTTISVTASVTIALVPPDQPEVQLSSM | | |
| | TMDARLSAKMALRGKALRTQLDLRRFRI | | |
| | YSNHSALESLALIPLQAPLKTMLQIGVMP | | |
| | MLNERTWRGVQIPLPEGINFVHEVVTNH | | |
| | AGFLTIGADLHFAKGLREVIEKNRPADVR | | |
| | ASTAPTPSTAAV | | |
| | (SEQ ID NO: 15)/P55065 | | |
| | (mouse) | | |
| Complement | 721 | 1.13 | C4B participates in the |
| C4-B (C4B) | (human) | | complement system, is |
| | MRLLWGLIWASSFFTLSLQKPRLLLFSPS | | derived from human |
| | VVHLGVPLSVGVQLQDVPRGQVVKGSV | | leukocyte antigen |
| | FLRNPSRNNVPCSPKVDFTLSSERDFAL | | (HLA), and functions |
| | LSLQVPLKDAKSCGLHQLLRGPEVQLVA | | in immunity (Agarwal |
| | HSPWLKDSLSRTTNIQGINLLFSSRRGHL | | V, Talens S, Grandits |
| | FLQTDQPIYNPGQRVRYRVFALDQKMR | | A M, Blom A M. J Biol |
| | PSTDTITVMVENSHGLRVRKKEVYMPSS | | Chem. 2015; 290(30): |
| | IFQDDFVIPDISEPGTWKISARFSDGLES | | 18333-18342). |
| | NSSTQFEVKKYVLPNFEVKITPGKPYILT | | |
| | VPGHLDEMQLDIQARYIYGKPVQGVAYV | | |
| | RFGLLDEDGKKTFFRGLESQTKLVNGQS | | |
| | HISLSKAEFQDALEKLNMGITDLQGLRLY | | |
| | VAAAIIESPGGEMEEAELTSWYFVSSPF | | |
| | SLDLSKTKRHLVPGAPFLLQALVREMSG | | |
| | SPASGIPVKVSATVSSPGSVPEVQDIQQ | | |
| | NTDGSGQVSIPIIIPQTISELQLSVSAGSP | | |
| | HPAIARLTVAAPPSGGPGFLSIERPDSRP | | |
| | PRVGDTLNLNLRAVGSGATFSHYYYMIL | | |
| | SRGQIVFMNREPKRTLTSVSVFVDHHLA | | |
| | PSFYFVAFYYHGDHPVANSLRVDVQAG | | |
| | ACEGKLELSVDGAKQYRNGESVKLHLET | | |
| | DSLALVALGALDTALYAAGSKSHKPLNM | | |
| | GKVFEAMNSYDLGCGPGGGDSALQVF | | |
| | QAAGLAFSDGDQWTLSRKRLSCPKEKT | | |
| | TRKKRNVNFQKAINEKLGQYASPTAKRC | | |
| | CQDGVTRLPMMRSCEQRAARVQQPDC | | |
| | REPFLSCCQFAESLRKKSRDKGQAGLQ | | |
| | RALEILQEEDLIDEDDIPVRSFFPENWLW | | |
| | RVETVDRFQILTLWLPDSLTTWEIHGLSL | | |
| | SKTKGLCVATPVQLRVFREFHLHLRLPM | | |
| | SVRRFEQLELRPVLYNYLDKNLTVSVHV | | |
| | SPVEGLCLAGGGGLAQQVLVPAGSARP | | |
| | VAFSVVPTAATAVSLKVVARGSFEFPVG | | |
| | DAVSKVLQIEKEGAIHREELVYELNPLDH | | |
| | RGRTLEIPGNSDPNMIPDGDFNSYVRVT | | |
| | ASDPLDTLGSEGALSPGGVASLLRLPRG | | |
| | CGEQTMIYLAPTLAASRYLDKTEQWSTL | | |
| | PPETKDHAVDLIQKGYMRIQQFRKADGS | | |
| | YAAWLSRGSSTWLTAFVLKVLSLAQEQV | | |
| | GGSPEKLQETSNWLLSQQQADGSFQDL | | |
| | SPVIHRSMQGGLVGNDETVALTAFVTIAL | | |
| | HHGLAVFQDEGAEPLKQRVEASISKASS | | |
| | FLGEKASAGLLGAHAAAITAYALTLTKAP | | |
| | ADLRGVAHNNLMAMAQETGDNLYWGS | | |
| | VTGSQSNAVSPTPAPRNPSDPMPQAPA | | |
| | LWIETTAYALLHLLLHEGKAEMADQAAA | | |
| | WLTRQGSFQGGFRSTQDTVIALDALSAY | | |
| | WIASHTTEERGLNVTLSSTGRNGFKSHA | | |
| | LQLNNRQIRGLEEELQFSLGSKINVKVG | | |
| | GNSKGTLKVLRTYNVLDMKNTTCQDLQI | | |
| | EVTVKGHVEYTMEANEDYEDYEYDELP | | |
| | AKDDPDAPLQPVTPLQLFEGRRNRRRR | | |
| | EAPKVVEEQESRVHYTVCIWRNGKVGL | | |
| | SGMAIADVTLLSGFHALRADLEKLTSLSD | | |
| | RYVSHFETEGPHVLLYFDSVPTSRECVG | | |
| | FEAVQEVPVGLVQPASATLYDYYNPERR | | |
| | CSVFYGAPSKSRLLATLCSAEVCQCAEG | | |
| | KCPRQRRALERGLQDEDGYRMKFACYY | | |
| | PRVEYGFQVKVLREDSRAAFRLFETKIT | | |
| | QVLHFTKDVKAAANQMRNFLVRASCRL | | |
| | RLEPGKEYLIMGLDGATYDLEGHPQYLL | | |
| | DSNSWIEEMPSERLCRSTRQRAACAQL | | |
| | NDFLQEYGTQGCQV | | |
| | (SEQ ID NO: 16)/P01029 | | |
| | (mouse) | | |
| Beta-enolase | 2027 | 1.09 | ENO3 is found in |
| (ENO3) | (human) | | skeletal muscle cells |
| | MAMQKIFAREILDSRGNPTVEVDLHTAK | | and could play a role |
| | GRFRAAVPSGASTGIYEALELRDGDKGR | | in muscle development |
| | YLGKGVLKAVENINNTLGPALLQKKLSVV | | and regeneration |
| | DQEKVDKFMIELDGTENKSKFGANAILG | | (Peshavaria M, Day |
| | VSLAVCKAGAAEKGVPLYRHIADLAGNP | | I N. Biochem J. 1993; |
| | DLILPVPAFNVINGGSHAGNKLAMQEFMI | | 292 (Pt 3):701-704). |
| | LPVGASSFKEAMRIGAEVYHHLKGVIKA | | |
| | KYGKDATNVGDEGGFAPNILENNEALEL | | |
| | LKTAIQAAGYPDKVVIGMDVAASEFYRN | | |
| | GKYDLDFKSPDDPARHITGEKLGELYKS | | |
| | FIKNYPVVSIEDPFDQDDWATWTSFLSG | | |
| | VNIQIVGDDLTVTNPKRIAQAVEKKACNC | | |
| | LLLKVNQIGSVTESIQACKLAQSNGWGV | | |
| | MVSHRSGETEDTFIADLVVGLCTGQIKT | | |
| | GAPCRSERLAKYNQLMRIEEALGDKAIF | | |
| | AGRKFRNPKAK | | |
| | (SEQ ID NO: 17)/P21550 | | |
| | (mouse) | | |
| Ig heavy | /P06330 | 1.08 | Unknown |
| chain V | (mouse) | | |
| region AC38 | EVQLQQSGPELVKPGASVKISCKASGYT | | |
| 205.12 (NAN) | FTDYYMNVWKQSHGKSLEWIGDINPNN | | |
| | GGTSYNQKFKGKATLTVDKSSSATYMEL | | |
| | RSLTSEDSAVYYCARGYGYDPFDVWGT | | |
| | GTTVTVSS | | |
| | (SEQ ID NO: 18) | | |

TABLE 2

| Name | UniprotKB ID | Functions |
|---|---|---|
| Platelet | P16109 | Stored in |
| bound P- | (human) | alpha |
| selectin | | granules |
| (CD62P) | | of |
| Cell | MANCQIAILYQRFQRVVFGISQLLCFSALI | platelets |
| Surface | SELTNQKEVAAWTYHYSTKAYSWNISRKYC | and |
| Expres- | QNRYTDLVAIQNKNEIDYLNKVLPYYSSYY | released |
| sion | WIGIRKNNKTWTWVGTKKALTNEAENWADN | upon ac- |
| | EPNNKRNNEDCVEIYIKSPSAPGKWNDEHC | tivation |
| | LKKKHALCYTASCQDMSCSKQGECLETIGN | |
| | YTCSCYPGFYGPECEYVRECGELELPQHVL | |
| | MNCSHPLGNFSFNSQCSFHCTDGYQVNGPS | |
| | KLECLASGIWTNKPPQCLAAQCPPLKIPER | |
| | GNMTCLHSAKAFQHQSSCSFSCEEGFALVG | |
| | PEVVQCTASGVWTAPAPVCKAVQCQHLEAP | |
| | SEGTMDCVHPLTAFAYGSSCKFECQPGYRV | |
| | RGLDMLRCIDSGHWSAPLPTCEAISCEPLE | |
| | SPVHGSMDCSPSLRAFQYDTNCSFRCAEGF | |
| | MLRGADIVRCDNLGQWTAPAPVCQALQCQD | |
| | LPVPNEARVNCSHPFGAFRYQSVCSFTCNE | |
| | GLLLVGASVLQCLATGNWNSVPPECQAIPC | |
| | TPLLSPQNGTMTCVQPLGSSSYKSTCQFIC | |
| | DEGYSLSGPERLDCTRSGRWTDSPPMCEAI | |
| | KCPELFAPEQGSLDCSDTRGEFNVGSTCHF | |
| | SCDNGFKLEGPNNVECTTSGRWSATPPTCK | |
| | GIASLPTPGLQCPALTTPGQGTMYCRHHPG | |
| | TFGFNTTCYFGCNAGFTLIGDSTLSCRPSG | |
| | QWTAVTPACRAVKCSELHVNKPIAMNCSNL | |
| | WGNFSYGSICSFHCLEGQLLNGSAQTACQE | |
| | NGHWSTTVPTCQAGPLTIQEALTYFGGAVA | |
| | STIGLIMGGTLLALLRKRFRQKDDGKCPLN | |
| | PHSHLGTYGVFTNAAFDPSP | |
| | (SEQ ID NO: 19) | |
| Integrin | P08514 | Integrin |
| alpha- | (human) | alpha- |
| IIb/ | MARALCPLQALWLLEWVLLLLGPCAAPPAW | IIb/ |
| beta-3 | ALNLDPVQLTFYAGPNGSQFGFSLDFHKDS | beta-3 |
| (or | HGRVAIVVGAPRTLGPSQEETGGVFLCPWR | acts as a |
| GP2B-3A | AEGGQCPSLLFDLRDETRNVGSQTLQTFKA | receptor |
| or CD61) | RQGLGASVVSWSDVIVACAPWQHWNVLEKT | for fi- |
| Cell | EEAEKTPVGSCFLAQPESGRRAEYSPCRGN | bronectin, |
| surface | TLSRIYVENDFSWDKRYCEAGFSSVVTQAG | fibrino- |
| expres- | ELVLGAPGGYYFLGLLAQAPVADPFSSYRP | gen, |
| sion | GILLWHVSSQSLSFDSSNPEYFDGYWGYSV | plasmino- |
| | AVGEFDGDLNTTEYVVGAPTWSWTLGAVEI | gen, pro- |
| | LDSYYQRLHRLRGEQMASYFGHSVAVTDVN | thrombin, |
| | GDGRHDLLVGAPLYMESRADRKLAEVGRVY | thrombo- |
| | LFLQPRGPHALGAPSLLLTGTQLYGRFGSA | spondin |
| | IAPLGDLDRDGYNDPAVAAPYGGPSGRGQV | and vitro- |
| | LVFLGQSEGLRSRPSQVLDSPFPTGSAFGF | nectin. |
| | SLRGAVDIDDNGYPDLIVGAYGANQVAVYR | After its |
| | AQPVVKASVQLLVQDSLNPAVKSCVLPQTK | activa- |
| | TPVSCFNIQMCVGATGHNIPQKLSLNAELQ | tion, it |
| | LDRQKPRQGRRVLLLGSQQAGTTLNLDLGG | causes |
| | KHSPICHTTMAFLRDEADFRDKLSPIVLSL | platelet/ |
| | NVSLPPTEAGMAPAVVLHGDTHVQEQTRIV | platelet |
| | LDCGEDDVCVPQLQLTASVTGSPLLVGADN | interac- |
| | VLELQMDAANEGEGAYEAELAVHLPQGAHY | tion |
| | MRALSNVEGFERLICNQKKENETRVVLCEL | through |
| | GNPMKKNAQIGPIMLVSVGNLEEAGESVSF | binding |
| | QLQIRSKNSQNPNSKIVLLDVPVRAEAQVE | soluble |
| | LRGNSFPASLVVAAEEGEREQNSLDSWGPK | fibrino- |
| | VEHTYELHNNGPGTVNGLHLSPHLPGQSQP | gen. This |
| | SDLLYILDIQPQGGLQCFPQPPVNPLKVDW | leads to |
| | GLPIPSPSPIHPAHHKRDRRQIFLPEPEQP | platelet |
| | SRLQDPVLVSCDSAPCTVVQCDLQEMARGQ | aggrega- |
| | RAMVTVLAFLWLPSLYQRPLDQFVLQSHAW | tion. |
| | FNVSSLPYAVPPLSLPRGEAQVWTQLLRAL | |
| | EERAIPIWWVLVGVLGGLLLLTILVLAMWK | |
| | VGFFKRNRPPLEEDDEEGE | |
| | (SEQ ID NO: 22) | |

In some embodiments, methods of measuring and/or monitoring Klotho activity in an animal (e.g. a human) are provided. This can be achieved, for example, by measuring the quantity or activity of one or more of the proteins listed in Table 1 or Table 2. In some embodiments, more than one protein quantity or activity can be used, e.g., 2, 3, 4, 5, or more proteins from Table 1 or Table 2. In some embodiments, the protein amount can be measured in a sample from the animal. Protein quantity can be measured in the sample as desired. In some embodiments, the protein can be measured by detection with an antibody or other reagent that specifically binds to the protein of Table 1 or Table 2. A number of immunoassay formats are known and can be used. For example, a direct or competitive immunoassay can be used. In some embodiments, the protein can be detected and quantified using mass spectrometry.

In other embodiments, protein activity can be detected. Methods of detecting protein activity will depend on the protein of Table 1 or Table 2 that is detected. In embodiments in which the target protein of Table 1 or 2 is an enzyme that has activity on a substrate, one can provide the substrate and detect activity by monitoring conversion of the substrate to a product, of a set time period, for example.

A sample can be obtained from a patient, e.g., a biopsy, from an animal, such as an animal model, or from cultured cells, e.g., a cell line or cells removed from a patient and grown in culture for observation. Biological samples include tissues and bodily fluids, e.g., blood, blood fractions, lymph, saliva, urine, feces, etc. In some embodiments, the sample is enriched for platelets or comprises purified platelets. See, e.g., O. Leiter et al., Stem Cell Reports 12, 667-679 (2019); L. C. Burzynski, N. Pugh, M. C. Clarke, Platelet Isolation and Activation Assays. Bio-protocol 9, e3405 (2019).

In some embodiments, the animal assayed for quantity or activity of a protein from Table 1 or 2 has not yet been administered Klotho (or has not be administered Klotho recently, e.g., within the past two weeks). In some of these embodiments, one can assay the quantity or activity of a protein of Table 1 or Table 2 to identify one or more animals (e.g., humans) that are likely to be responsive to Klotho treatment. For example, in some embodiments, an animal will be selected as likely to be responsive to Klotho treatment of the animal has a quantity or activity of a protein of Table 1 or 2 below a control value, which can be used to predict those animals that will most benefit from increased quantity or activity of those proteins.

In some embodiments, the control value can be determined as representative of an average population value, or for example, a statistically lower or higher value (e.g., one or two standard deviations from the average), or representing a diseased state. In some embodiments, more than one control value can be compared to the determined quantity of the sample. For example, one can use both baseline and “challenged” levels. An example of a challenged level might be for example a representative level while performing a cognitive task or participating in physical exertion. Similarly, in some embodiments, more than one sample is taken from the animal, wherein in some embodiments the animal is cognitively or physically challenged whereas a second sample is taken from the animal is in a rest state, and both can be compared to corresponding control values. Levels or actions of platelets factors or other factors in Tables 1 or 2 can be informative at both baseline and with challenge to assess degree of platelet activation.

In some embodiments, the animal assayed for quantity or activity of a protein from Table 1 or 2 has been administered Klotho, for example within the past month, past two weeks, past week, past 1 or 2 days, past 12, 8, 4, 2, or 1 hour. This assay can be used, for example, to determine that the Klotho protein administered had the expected activity, regardless of whether or not the ultimate desired effect (improved cognition, for example) was attained. This can be useful for example, to confirm the Klotho protein was active, for example in clinical and preclinical trials and analysis, allowing one to distinguish, for example, poor results from inactive protein compared to poor results from active protein. In some embodiments, multiple different doses can be administered to the animal and the quantity or activity of the protein of Table 1 or 2 can be determined to assist in determining preferred or optimal Klotho dosage.

Klotho protein that is administered can be any animal (e.g., human) Klotho protein, functional variant or fragment thereof. Exemplary Klotho proteins are described in, e.g., U.S. Pat. No. 10,300,117. Klotho is a pleiotropic protein and an aging regulator that circulates throughout the body and brain (Imura et al. (2004) FEBS Letters 565:143; Kurosu et al. (2005) Science 309:1829). Human Klotho is described in GenBank Accession No. NC_000013 and Uniprot Accession No. Q9UEF7. A number of species homologs exist, including mouse and rat Klotho which share 86% and 85% identity with the human Klotho polypeptide, which is shown as SEQ ID NO:1.

```
SEQ ID NO: 1:
Met Pro Ala Ser Ala Pro Pro Arg Arg Pro Arg
1 5 10
Pro Pro Pro Pro Ser Leu Ser Leu Leu Leu Val
                        15 20
Leu Leu Gly Leu Gly Gly Arg Arg Leu Arg Ala
                25 30
Glu Pro Gly Asp Gly Ala Gln Thr Trp Ala Arg
        35 40
Phe Ser Arg Pro Pro Ala Pro Glu Ala Ala Gly
45 50 55
Leu Phe Gln Gly Thr Phe Pro Asp Gly Phe Leu
                                60 65
Trp Ala Val Gly Ser Ala Ala Tyr Gln Thr Glu
                        70 75
Gly Gly Trp Gln Gln His Gly Lys Gly Ala Ser
                80 85
Ile Trp Asp Thr Phe Thr His His Pro Leu Ala
        90 95
Pro Pro Gly Asp Ser Arg Asn Ala Ser Leu Pro
100 105 110
Leu Gly Ala Pro Ser Pro Leu Gln Pro Ala Thr
                                115 120
Gly Asp Val Ala Ser Asp Ser Tyr Asn Asn Val
                        125 130
Phe Arg Asp Thr Glu Ala Leu Arg Glu Leu Gly
                135 140
Val Thr His Tyr Arg Phe Ser Ile Ser Trp Ala
        145 150
Arg Val Leu Pro Asn Gly Ser Ala Gly Val Pro
155 160 165
Asn Arg Glu Gly Leu Arg Tyr Tyr Arg Arg Leu
                                170 175
Leu Glu Arg Leu Arg Glu Leu Gly Val Gln Pro
                        180 185
Val Val Thr Leu Tyr His Trp Asp Leu Pro Gln
                190 195
Arg Leu Gln Asp Ala Tyr Gly Gly Trp Ala Asn
        200 205
Arg Ala Leu Ala Asp His Phe Arg Asp Tyr Ala
210 215 220
Glu Leu Cys Phe Arg His Phe Gly Gly Gln Val
                                225 230
Lys Tyr Trp Ile Thr Ile Asp Asn Pro Tyr Val
                        235 240
Val Ala Trp His Gly Tyr Ala Thr Gly Arg Leu
                245 250
Ala Pro Gly Ile Arg Gly Ser Pro Arg Leu Gly
        255 260
Tyr Leu Val Ala His Asn Leu Leu Leu Ala His
265 270 275
Ala Lys Val Trp His Leu Tyr Asn Thr Ser Phe
                                280 285
Arg Pro Thr Gln Gly Gly Gln Val Ser Ile Ala
                        290 295
Leu Ser Ser His Trp Ile Asn Pro Arg Arg Met
                300 305
Thr Asp His Ser Ile Lys Glu Cys Gln Lys Ser
        310 315
Leu Asp Phe Val Leu Gly Trp Phe Ala Lys Pro
320 325 330
Val Phe Ile Asp Gly Asp Tyr Pro Glu Ser Met
                                335 340
Lys Asn Asn Leu Ser Ser Ile Leu Pro Asp Phe
                        345 350
Thr Glu Ser Glu Lys Lys Phe Ile Lys Gly Thr
                355 360
Ala Asp Phe Phe Ala Leu Cys Phe Gly Pro Thr
        365 370
Leu Ser Phe Gln Leu Leu Asp Pro His Met Lys
375 380 385
Phe Arg Gln Leu Glu Ser Pro Asn Leu Arg Gln
                                390 395
Leu Leu Ser Trp Ile Asp Leu Glu Phe Asn His
                        400 405
Pro Gln Ile Phe Ile Val Glu Asn Gly Trp Phe
                410 415
Val Ser Gly Thr Thr Lys Arg Asp Asp Ala Lys
        420 425
Tyr Met Tyr Tyr Leu Lys Lys Phe Ile Met Glu
430 435 440
Thr Leu Lys Ala Ile Lys Leu Asp Gly Val Asp
                                445 450
Val Ile Gly Tyr Thr Ala Trp Ser Leu Met Asp
                        455 460
Gly Phe Glu Trp His Arg Gly Tyr Ser Ile Arg
                465 470
Arg Gly Leu Phe Tyr Val Asp Phe Leu Ser Gln
        475 480
Asp Lys Met Leu Leu Pro Lys Ser Ser Ala Leu
485 490 495
Phe Tyr Gln Lys Leu Ile Glu Lys Asn Gly Phe
                                500 505
Pro Pro Leu Pro Glu Asn Gln Pro Leu Glu Gly
                        510 515
Thr Phe Pro Cys Asp Phe Ala Trp Gly Val Val
                520 525
Asp Asn Tyr Ile Gln Val Asp Thr Thr Leu Ser
        530 535
Gln Phe Thr Asp Leu Asn Val Tyr Leu Trp Asp
540 545 550
Val His His Ser Lys Arg Leu Ile Lys Val Asp
                                555 560
Gly Val Val Thr Lys Lys Arg Lys Ser Tyr Cys
                        565 570
Val Asp Phe Ala Ala Ile Gln Pro Gln Ile Ala
                575 580
Leu Leu Gln Glu Met His Val Thr His Phe Arg
        585 590
Phe Ser Leu Asp Trp Ala Leu Ile Leu Pro Leu
595 600 605
Gly Asn Gln Ser Gln Val Asn His Thr Ile Leu
                                610 615
Gln Tyr Tyr Arg Cys Met Ala Ser Glu Leu Val
                        620 625
Arg Val Asn Ile Thr Pro Val Val Ala Leu Trp
                630 635
Gln Pro Met Ala Pro Asn Gln Gly Leu Pro Arg
        640 645
Leu Leu Ala Arg Gln Gly Ala Trp Glu Asn Pro
650 655 660
Tyr Thr Ala Leu Ala Phe Ala Glu Tyr Ala Arg
                                665 670
Leu Cys Phe Gln Glu Leu Gly His His Val Lys
                        675 680
Leu Trp Ile Thr Met Asn Glu Pro Tyr Thr Arg
                685 690
Asn Met Thr Tyr Ser Ala Gly His Asn Leu Leu
        695 700
Lys Ala His Ala Leu Ala Trp His Val Tyr Asn
705 710 715
Glu Lys Phe Arg His Ala Gln Asn Gly Lys Ile
                                720 725
Ser Ile Ala Leu Gln Ala Asp Trp Ile Glu Pro
                        730 735
Ala Cys Pro Phe Ser Gln Lys Asp Lys Glu Val
                740 745
Ala Glu Arg Val Leu Glu Phe Asp Ile Gly Trp
        750 755
Leu Ala Glu Pro Ile Phe Gly Ser Gly Asp Tyr
760 765 770
Pro Trp Val Met Arg Asp Trp Leu Asn Gln Arg
                                775 780
Asn Asn Phe Leu Leu Pro Tyr Phe Thr Glu Asp
                        785 790
Glu Lys Lys Leu Ile Gln Gly Thr Phe Asp Phe
                795 800
Leu Ala Leu Ser His Tyr Thr Thr Ile Leu Val
        805 810
Asp Ser Glu Lys Glu Asp Pro Ile Lys Tyr Asn
815 820 825
Asp Tyr Leu Glu Val Gln Glu Met Thr Asp Ile
                                830 835
Thr Trp Leu Asn Ser Pro Ser Gln Val Ala Val
                        840 845
Val Pro Trp Gly Leu Arg Lys Val Leu Asn Trp
                850 855
Leu Lys Phe Lys Tyr Gly Asp Leu Pro Met Tyr
        860 865
Ile Ile Ser Asn Gly Ile Asp Asp Gly Leu His
870 875 880
Ala Glu Asp Asp Gln Leu Arg Val Tyr Tyr Met
                                885 890
Gln Asn Tyr Ile Asn Glu Ala Leu Lys Ala His
                        895 900
Ile Leu Asp Gly Ile Asn Leu Cys Gly Tyr Phe
                905 910
Ala Tyr Ser Phe Asn Asp Arg Thr Ala Pro Arg
        915 920
Phe Gly Leu Tyr Arg Tyr Ala Ala Asp Gln Phe
925 930 935
Glu Pro Lys Ala Ser Met Lys His Tyr Arg Lys
                                940 945
Ile Ile Asp Ser Asn Gly Phe Pro Gly Pro Glu
                        950 955
Thr Leu Glu Arg Phe Cys Pro Glu Glu Phe Thr
                960 965
Val Cys Thr Glu Cys Ser Phe Phe His Thr Arg
        970 975
Lys Ser Leu Leu Ala Phe Ile Ala Phe Leu Phe
980 985 990
Phe Ala Ser Ile Ile Ser Leu Ser Leu Ile Phe
                                995 1000
Tyr Tyr Ser Lys Lys Gly Arg Arg Ser Tyr Lys
                        1005 1010
```

Klotho exists naturally in a transmembrane form that can be cleaved such that the extracellular portion (amino acids 34-979) is released as a hormone (Shiraki-lika et al. (1998) FEBS Letters 424:6). Klotho also has a splice variant that results in a 549 amino acid secreted form of the protein that is also functional (Wang and Sun (2009) Ageing Res. Rev. 8:43). Both cleaved and secreted klotho are soluble and functional in the body, but have a sequence variation at the C-terminal end due to the splice variation. Amino acids 535-549 are DTTLSQFTDLNVYLW (SEQ ID NO:20) for cleaved, soluble human Klotho and SQLTKPISSLTKPYH (SEQ ID NO:21) for spliced, soluble human Klotho. Full length soluble Klotho includes two conserved domains (KL1 and KL2) with homology to beta glycosidase proteins. The conserved beta-glucosidase/6-phospho-beta-glucosidase/beta-galactosidase motif spans 62-497 in the human protein and 64-499 in the mouse. The conserved KL1 sequence is described in Chateau et al. (2010) Aging 2:567 and Matsumura et al. (1998) Biochem Biophys Res Commun, and comprises amino acids 34-549 of the human Klotho protein, with the glycosyl hydrolase consensus region spanning amino acids 57-506 of the human Klotho protein (59-508 of the mouse). Klotho does not have beta-glycosidase activity, but shows some beta-glucuronidase activity.

Klotho suppresses insulin and wnt signaling, regulates ion channels and their transport, and promotes function of FGF23. See, e.g., Chang et al. (2005) Science 310:490; Imura et al. (2007) Science 316:1615; Kurosu (2005); Liu et al. (2007) Science 317:803; and Urakawa et al. (2006) Nature 444:770).

In mice, transgenic overexpression of klotho extends lifespan and associates with better cognitive functions in the normal and diseased brain (Kurosu (2005); Dubal et al. (2014) Cell Reports 7:1065; Dubal et al. (2015) J. Neuroscience). In humans, a single allele of the KL-VS variant of the KLOTHO gene, which increases secreted klotho promotes longevity (Arking et al., 2002; Arking et al., 2005; Invidia et al., 2010) and also associates with better baseline cognitive functions in aging populations. See, e.g., Arking et al. (2002) PNAS 99:856; Arking et al. (2005) Circ. Res. 96:412; Dubal et al (2014) Cell Reports 7:1065; Yokoyama et al. (2015) Ann. Clin. Translational Neurology 2:215.

Klotho polypeptides that can be used for administration include species homologs (e.g., non-human primate, mouse, rat), allelic variants, functional fragments, and functional variants of the wild type sequence that retain cognition improving activity. Examples include secreted Klotho, fragments comprising the KL1 domain, fragments comprising the KL2 domain, fragments comprising the KL1 and KL2 domains, variants comprising the KL1 domain with at least one (e.g., 1-20, 5-50, 25-100) non-conserved amino acid in the KL1 domain substituted with a different amino acid or deleted, variants comprising the KL2 domain with at least one non-conserved amino acid in the KL2 domain substituted with a different amino acid.

Functional fragments of the Klotho polypeptide that can be used as described herein include the extracellular domain (e.g., corresponding to or substantially identical or similar to amino acids 34-979 of human Klotho), secreted Klotho (e.g., corresponding to or substantially identical or similar to 549 amino acid form), a KL1 domain (e.g., corresponding to or substantially identical or similar to amino acids 34-549 of human Klotho), a glycosyl hydrolase consensus sequence (e.g., corresponding to or substantially identical or similar to amino acids 57-506 of human Klotho), or a beta-glucosidase/6-phospho-beta-glucosidase/beta-galactosidase consensus sequence (e.g., corresponding to or substantially identical or similar to amino acids 62-497 of human Klotho). In some embodiments, the Klotho polypeptide comprises or is substantially identical or similar to amino acids 34-549 of human Klotho. In some embodiments, the Klotho polypeptide is part of a larger fusion protein. In some embodiments, the fusion protein comprises the Klotho polypeptide as described herein and further comprises no more than 100, 75, 50, or 30 additional amino acids. In some embodiments, the Klotho polypeptide is not fused to a Fibroblast growth factor (FGF). In some embodiments, the Klotho polypeptide comprises (e.g., is fused to) an affinity tag (e.g., a histidine tag) or a conjugate to increase stability or half-life in vivo.

A functional variant or fragment of Klotho is a variant or fragment that retains any klotho activity, e.g., at least 20%, 30%, 40%, 50%, 60%, 70%, 80%, 90%, or 100% of the level of any activity of soluble klotho. Soluble klotho activities include those described above, and include binding FGF-23, binding to FGFR1c, beta-glucuronidase activity, suppression of wnt signaling, suppression of insulin signaling, suppression of TFG-beta 1 activity, increasing GluN2B expression and/or synaptic localization, c-fos induction. Additional Klotho activities include causing changes in magnetic resonance imaging (MRI) brain scans, e.g., functional MRI, electroencephalograph (EEG), and transcranial magnetic and electrical stimulation (TMS and TES); and improved performance in neuropsychologic testing and cognitive ability.

In some embodiments, the Klotho polypeptide is administered in a pharmaceutical composition with a physiologically (i.e., pharmaceutically) acceptable carrier. The term “carrier” refers to a typically inert substance used as a diluent or vehicle for a diagnostic or therapeutic agent. The term also encompasses a typically inert substance that imparts cohesive qualities to the composition. Physiologically acceptable carriers can be liquid, e.g., physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions of the present invention (See, e.g., Remington's Pharmaceutical Sciences, 17^{th} ed., 1989).

The Klotho compositions can be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions, such as a stabilizer for lyophilized antibody compositions.

Dosage forms can be prepared for mucosal (e.g., nasal, sublingual, vaginal, buccal, or rectal), parenteral (e.g., subcutaneous, intravenous, intramuscular, or intraarterial injection, either bolus or infusion), oral, or transdermal administration to a patient. Examples of dosage forms include, but are not limited to: dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Injectable compositions can comprise a solution of the Klotho polypeptide suspended in an acceptable carrier, such as an aqueous carrier. Any of a variety of aqueous carriers can be used, e.g., water, buffered water, 0.4% saline, 0.9% isotonic saline, 0.3% glycine, 5% dextrose, and the like, and may include glycoproteins for enhanced stability, such as albumin, lipoprotein, globulin, etc. In some embodiments, normal buffered saline (135-150 mM NaCl) is used. The compositions can contain pharmaceutically acceptable auxiliary substances to approximate physiological conditions, such as pH adjusting and buffering agents, tonicity adjusting agents, wetting agents, e.g., sodium acetate, sodium lactate, sodium chloride, potassium chloride, calcium chloride, sorbitan monolaurate, triethanolamine oleate, etc.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In some embodiments, the composition is administered by intravenous infusion, topically, intraperitoneally, intravesically, or intrathecally. The Klotho polypeptide formulation can be provided in unit-dose or multi-dose sealed containers, such as ampoules and vials.

The Klotho polypeptide composition, alone or in combination with other suitable components, can be made into aerosol formulations (“nebulized”) to be administered via inhalation. Aerosol formulations can be placed into pressurized acceptable propellants, such as dichlorodifluoromethane, propane, and nitrogen.

The pharmaceutical preparation can be packaged or prepared in unit dosage form. In such form, the preparation is subdivided into unit doses containing appropriate quantities of the active component, e.g., according to the dose of Klotho polypeptide. The unit dosage form can be a packaged preparation, the package containing discrete quantities of preparation. The composition can, if desired, also contain other compatible therapeutic agents. In some embodiments, the Klotho polypeptide composition can be formulated in a kit for administration.

In some embodiments, a pharmaceutical composition comprising a klotho polypeptide is administered orally. In some embodiments, a pharmaceutical composition comprising a klotho polypeptide is administered mucosally, e.g., nasally. In some embodiments, a pharmaceutical composition comprising a klotho polypeptide is administered by injection, e.g., subcutaneous, intraperitoneal, intravenous, or intramuscular. In some embodiments, a pharmaceutical composition comprising a klotho polypeptide is administered by infusion, e.g., using a reservoir or osmotic minipump.

An example of administration of a pharmaceutical composition includes storing the Klotho polypeptide at 10 mg/ml in sterile isotonic aqueous saline solution at 4° C., and diluting it in an appropriate solution for injection prior to administration to the patient. In some embodiments, the Klotho polypeptide composition can be administered by intravenous infusion over the course of 0.25-2 hours. In some embodiments, the administration procedure is via bolus injection.

In therapeutic use, the Klotho polypeptide can be administered at the initial dosage of about 0.1 μg/kg to about 1000 μg/kg daily and adjusted over time. A daily dose range of about 1 μg/kg to about 500 μg/kg, or about 10 μg/kg to about 100 μg/kg, or about 30 μg/kg to about 50 ug/kg can be used. The dosage is varied depending upon the requirements of the patient, the severity of the condition being treated, and the route of administration. For example, for injection of Klotho polypeptide, the effective dose is typically in the range of 10-100 μg/kg, while for direct delivery to the central nervous system (CNS), the effective dosage is lower, e.g., 5-30 μg/kg. For oral administration, the effective dose is higher, e.g., in the range of 50-10,000 μg/kg (e.g., 100 μg/kg-2 mg/kg). The dose is chosen in order to provide effective therapy for the patient. The dose may be repeated at an appropriate frequency which may be in the range of once or twice per day, once or twice per week to once every three months, depending on the pharmacokinetics of the Klotho polypeptide composition (e.g., half-life in the circulation) and the pharmacodynamic response (e.g., the duration of the therapeutic effect).

Administration can be periodic. Depending on the route of administration, the dose can be administered, e.g., once every 1, 3, 5, 7, 10, 14, 21, or 28 days or longer (e.g., once every 2, 3, 4, or 6 months). In some cases, administration is more frequent, e.g., 2 or 3 times per day. The patient can be monitored to adjust the dosage and frequency of administration depending on therapeutic progress and any adverse side effects, as will be recognized by one of skill in the art.

Dosages can be empirically determined considering the type and severity of cognitive condition diagnosed in a particular patient. The dose administered to a patient, in the context of the present disclosure, should be sufficient to affect a beneficial therapeutic response in the patient over time. The size of the dose will also be determined by the existence, nature, and extent of any adverse side-effects that accompany the administration of any particular composition in a particular patient.

In some embodiments, the Klotho polypeptide is linked to a stabilizing moiety such as PEG, glycosylation, or a liposome or other nanocarrier. U.S. Pat. Nos. 4,732,863 and 7,892,554 and Chattopadhyay et al. (2010) Mol Pharm 7:2194 describe methods for attaching a polypeptide to PEG, PEG derivatives, and nanoparticles (e.g., liposomes). Liposomes containing phosphatidyl-ethanolamine (PE) can be prepared by established procedures as described herein. The inclusion of PE provides an active functional site on the liposomal surface for attachment. In some embodiments, the Klotho polypeptide is linked to an affinity tag, e.g., a histidine tag (e.g., 4-16 histidine residues (SEQ ID NO: 23)), streptavidin, or an antibody target.

The Klotho polypeptide can also be formulated as a sustained-release preparation (e.g., in a semi-permeable matrices of solid hydrophobic polymers (e.g., polyesters, hydrogels (for example, poly (2-hydroxyethyl-methacrylate), or poly (vinylalcohol)), polylactides. The Klotho polypeptide can be entrapped in a nanoparticle prepared, for example, by coacervation techniques or by interfacial polymerization, for example, hydroxymethylcellulose or gelatin microcapsules and poly-(methylmethacylate) microcapsules, respectively, in colloidal drug delivery systems (for example, liposomes, albumin microspheres, microemulsions, nano-particles and nanocapsules) or in macroemulsions.

### EXAMPLE

Example 1

Methods

Mice

All studies were conducted in a blinded manner in C57BL/6 mice. Young mice and aged mice were obtained from The Jackson Laboratory and the National Institute on Aging (NIA) mouse colonies, respectively. Mice were randomly assigned to each group, and the experimenter was blinded to their treatment. Mice were kept on a 12-hr light/dark cycle with ad libitum access to food (Picolab Rodent Diet 20) and water. All studies were approved by the Institutional Animal Care and Use Committee of the University of California, San Francisco and conducted in compliance with NIH guidelines.

Plasma Profiling of Mouse Plasma

Mouse klotho (R&D, 1819-KL) was diluted in PBS (pH7.5) and administered as an i.p. injection at a volume of 10 ul/gram (adjusted to weight of mouse) at a dose of 10 μg/kg. All young male mice (4 months old) were injected with vehicle or klotho (n=10 mice per group). Four hours later, they explored a small Y-maze for 10 minutes and their brains were immediately harvested following anesthesia with avertin (i.p.). Whole blood was collected from the cardiac puncture route into EDTA-coated tubes (Sastedt), centrifugated with 10,000 rpm for 10 min and then plasma was transferred to a low-binding tube (Sastedt). Plasma samples were processed for analyzed by mass spectrometry at Biognosys, Zurich, Switzerland.

Cognitive Behavioral test

Mouse platelet factor 4 (PF4) (PROSPEC, chm-245) was diluted in PBS (pH7.5) and administered as an i.p. injection at a volume of 10 ul/gram (adjusted to weight of mouse) 1 h before each day of training and testing at a dose of (20 ug/kg). All female aged mice (18-21 months old, n=8 mice per group) were tested in two-trials Y-maze as described in Dellu F, Mayo W, Cherkaoui J, Le Moal M, Simon H. A two-trial memory task with automated recording: study in young and aged rats. Brain Res. 1992; 588(1):132-139. Briefly, mice underwent training by exploring the maze with a visual cue in one arm and another arm blocked off 16 h after training, mice underwent testing with the all three arms open (start arm, familiar arm, novel arm) and the time spent exploring the novel arm compared to the familiar arm, an index of memory, was tested.

Results

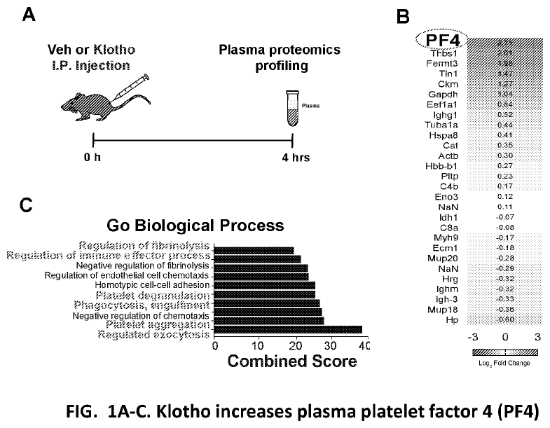

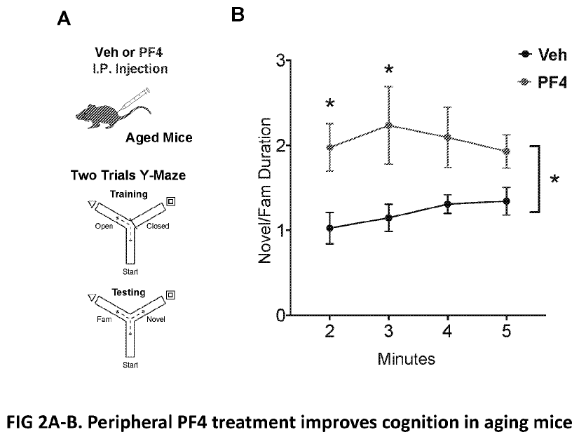

In order to assess how systemic elevation of klotho in the body sends a signal to boost cognition, we profiled plasma proteins following systemic klotho treatment (FIG. 1A-C). Klotho significantly increased several plasma platelet factors (FIG. 1B, Table 1), indicating a novel biologic action of klotho in inducing platelet activation and function (FIG. 1C). Klotho treatment most robustly increased platelet factor 4 (PF4) (FIG. 1B), a pleiotropic chemokine that increases with exercise and enhances neurogenesis (Leiter O, Seidemann S, Overall R W, et al. Exercise-Induced Activated Platelets Increase Adult Hippocampal Precursor Proliferation and Promote Neuronal Differentiation. Stem Cell Reports. 2019; 12(4):667-679). We then tested whether PF4 itself can affect cognition in the aging brain (FIG. 2A and B). Indeed, systemic treatment with recapitulated klotho-mediated improvement of cognition (FIG. 2B). These findings collectively suggest that klotho increases platelet factor, such as PF4— and factors such as PF4 induce cognitive enhancement.

### Example 2

### Results

### Systemic Klotho Treatment Activates Platelets

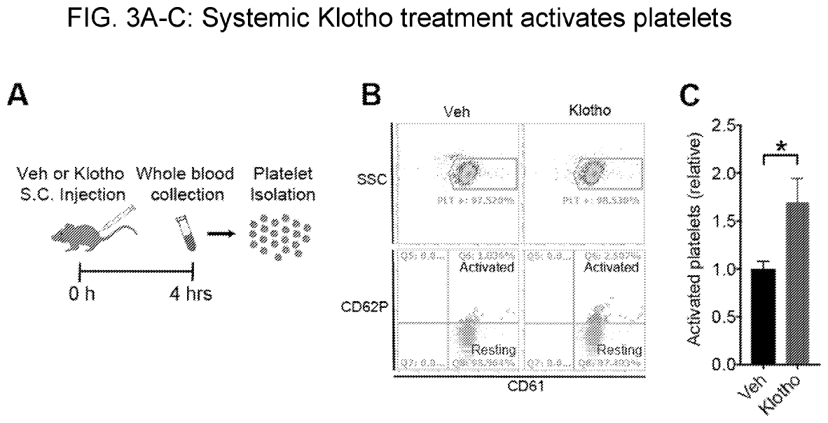

FIG. 3A-C provide data showing that Klotho treatment activates platelets. Paradigm for measuring platelet activation is depicted in FIG. 3A. Mice (age 5 months; n=8-9 mice per group) were treated with either Veh or Klotho (s.c., 10 μg/kg) followed by platelet isolation from whole blood and then platelet activation analysis by fluorescence activated single cell sorting (FACS) sorting with markers CD61 and CD62P.

FIG. 3B depicts flow cytometry plots from FACS sorting show a platelet populations. The upper graphs show density plots of the platelets, gated by SSC (for granularity) and CD61-positivity which both identify platelets from other blood cells. The lower graphs show dot plots of the percentage activated (CD61 and CD62P-positive) and resting (CD61-positive only) platelets.

FIG. 3C show quantification results of activated platelets in young mice following treatment with Veh or Klotho. Data are presented as means±SEM; *p<0.05 by two-tailed t-test.

### Materials and Methods

### Mice

All mice were on a congenic C57BL/6J background and kept on a 12-h light/dark cycle with ad libitum access to food and water. All studies were approved by the Institutional Animal Care and Use Committee of the University of California, San Francisco, and conducted in compliance with NIH guidelines.

### Drug Treatment

Mouse α-Klotho (R&D, 1819-KL) was diluted in PBS (pH. 7.5) and injected s.c. at a volume of 10 μl/gram (adjusted to weight of mouse) at a dose of 10 μg/kg 4 hrs prior to whole blood collection for platelet isolation and platelet activation assays. Recombinant proteins were used within one week of thawing from −80° C. stock solutions and stored at 4° C.

### Platelet Activation Assay by Flow Cytometry.

Platelet activation states were measured using flow cytometry as described (O. Leiter et al., Stem Cell Reports 12, 667-679 (2019); L. C. Burzynski, N. Pugh, M. C. Clarke, Platelet Isolation and Activation Assays. Bio-protocol 9, e3405 (2019)) with minor modifications. Briefly, whole blood via cardiac puncture was collected into a final concentration of 0.38% sodium citrate solution (pH 7) and then centrifuged at 200 g for 10 min at room temperature. Plasma was collected and transferred to a new tube with 1 ml of HBSS (with EDTA, pH 6.4) and then centrifuged at 1200 g for 20 min at room temperature. The platelet pellet was resuspended in HBSS (pH 6.4) and then stained with CD61-PE (Thermo Fischer) and CD62-alexa 647 (BD Bioscience) antibodies for 30 min at room temperature. FACS buffer in the amount of 1 mL (PBS with 1% BSA and 1% Sodium Azide (pH 6.4)) (2) was added. A total of 10,000 events were analyzed by flow cytometry at a flow rate of 25 μl/ml. Activated platelets were identified as CD62P-positive cells within the CD61-positive population.

mPF4 ELISA.

Enzyme-linked immunoabsorbant assay (ELISAs) (R&D Systems) were conducted according to the manufacturer's directions. Briefly, each plasma sample was diluted by 1:20 with ELISA buffer and analyzed for mPF4 per instructions.

The above examples are provided to illustrate the invention but not to limit its scope. Other variants of the invention will be readily apparent to one of ordinary skill in the art and are encompassed by the appended claims. All publications, databases, internet sources, patents, patent applications, and accession numbers cited herein are hereby incorporated by reference in their entireties for all purposes.

What is claimed is:

1. A method of assessing activity of a Klotho polypeptide in an animal, the method comprising
administering the Klotho polypeptide to the animal, and then
obtaining a sample from the animal and measuring a quantity or activity of any one or more polypeptide of Table 1 or Table 2.

2. The method of claim 1, wherein the sample comprises platelets.

3. The method of claim 2, wherein the obtaining comprises purifying platelets from blood from the animal.

4. The method of claim 1, comparing the quantity or activity to a control value.

5. The method of claim 4, further comprising administering an additional amount of the Klotho polypeptide to the animal if the quantity or activity is below the control value.

6. The method of claim 1, wherein the animal is a human.

7. A method of assessing an animal as a candidate for improved cognition treatment, the method comprising
obtaining a sample from the animal and measuring a quantity or activity of any one or more a polypeptide of Table 1 or Table 2;
comparing the quantity or activity to a control value; and then
administering an effective dose of a Klotho polypeptide or a protein comprising a polypeptide of Table 1 or Table 2 or a functional fragment or variant thereof to the animal to improve cognition in the animal.

18. The method of claim 7, wherein the sample comprises platelets.

19. The method of claim 8, wherein the obtaining comprises purifying platelets from blood from the animal.

10. The method of claim 7, further comprising after the administering,
obtaining a second sample from the animal and measuring the quantity or activity of any one or more the polypeptide of Table 1 or Table 2; and
comparing the quantity or activity from the second sample to a control value.

11. The method of claim 7, wherein the animal is a human.
